(19) United States
(12) Patent Application Publication
(10) Pub. No.: US 2026/0041552 A1
(43) Pub. Date: Feb. 12, 2026

(54) PROSTHETIC VALVES WITH RELEASABLE OUTER SKIRTS

(71) Applicant: EDWARDS LIFESCIENCES CORPORATION, Irvine, CA (US)

(72) Inventors: Tamir S. Levi, Zikhron Yaakov (IL); Noam Nir, Pardes-Hanna (IL); Michael Bukin, Pardes Hanna (IL); Eran Goldberg, Nesher (IL)

(73) Assignee: EDWARDS LIFESCIENCES CORPORATION, Irvine, CA (US)

(21) Appl. No.: 19/362,243

(22) Filed: Oct. 17, 2025

Related U.S. Application Data
(63) Continuation of application No. PCT/US2024/025653, filed on Apr. 22, 2024.

(51) Int. Cl. A61F 2/24

(52) U.S. Cl. CPC A61F 2/2418; A61F 2/2415; A61F 2220/0075; A61F 2230/0006; A61F 2240/001

(57) ABSTRACT

The present disclosure relates prosthetic valves and assemblies that include an outer skirt releasably coupled to the frame or to another skirt of the prosthetic valve. In an example, a prosthetic valve assembly includes a prosthetic valve comprising an annular frame and a first skirt attached to the frame, and a second skirt disposed radially outward to the first skirt and releasable coupled to the first skirt via at least one retaining suture attached to the frame and extending through inflow and outflow loops of the first and second skirts. The second skirt is configured to be released from the first skirt upon withdrawal of the at least one retaining suture from the inflow and outflow loops of both skirts.

### CROSS-REFERENCE TO RELATED APPLICATIONS

This application is a continuation of International Application No. PCT/US2024/025653, filed Apr. 22, 2024, which claims the benefit of U.S. Provisional Application No. 63/461,643, filed Apr. 25, 2023, which is incorporated by reference herein.

### FIELD

The present disclosure relates to prosthetic valves and prosthetic valve assemblies that include an outer skirt releasably coupled to the prosthetic valve, as well as methods of outer skirt removal from such prosthetic valves and assemblies.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic valve is mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath or a capsule of the delivery device so that the prosthetic valve can self-expand to its functional size. The prosthetic valve is then operational inside of the patient's heart and replaces the function of the native heart valve for the useful life of the prosthetic valve, which typically is about 15 to 25 years.

Most expandable, transcatheter heart valves comprise a radially expandable and compressible cylindrical metal frame and prosthetic leaflets mounted inside the frame. The frame can comprise a plurality of circumferentially extending rows of angled struts defining rows of open cells of the frame. The prosthetic valve can include an outer sealing member (also referred to as an “outer skirt”) affixed to an outer surface of the frame for sealing the prosthetic valve against tissue of the native heart valve. The outer skirt typically is attached to frame via sutures. The outer skirt typically encourages tissue ingrowth, which helps seal and anchor the prosthetic valve against the native annulus. The use of an outer skirt is particularly important for patients suffering from aortic stenosis, which causes calcification of the native aortic valve. The calcification can create an irregularly shaped surface on the native annulus tissue. Outer skirts have been designed to fill gaps between the frame of the prosthetic valve and the hardened calcium deposits of the surrounding tissue to establish a seal between the prosthetic valve and the native annulus.

### SUMMARY

In some patients, it may become desirable to remove or explant the prosthetic valve if the valve malfunctions, experiences wear and tear or deterioration from use over time, and/or as advances in prosthetic implant technologies are developed and an improved prosthetic valve device is desired. During explant procedures, the older prosthetic valve is removed such that a new prosthetic valve can be implanted within the diseased native valve. However, explantation of conventional prosthetic valves can be quite challenging due to the neointimal tissue formed between the outer skirt and the native annulus. Typically, the surgeon is required to cut the tissue surrounding the prosthetic valve in order to remove the prosthetic valve from the patient. The outer skirt typically is bounded on one side by the frame of the prosthetic valve and on other side by the native annulus, leaving little room for the surgeon to access the tissue that needs to be cut from the prosthetic valve. As can be appreciated, this is a delicate procedure and may involve significant risks to the patient. In some cases, for example, explantation of a prosthetic valve can result in trauma to the tissue of the left ventricular outflow tract.

Described herein are prosthetic valves having an outer skirt that is removable from the frame of the prosthetic valve, via removal of at least one retaining suture and/or by utilizing a cutting member disposed between the frame and the outer skirt. As such, devices and methods disclosed herein can, among other things, overcome one or more of the deficiencies of typical prosthetic valves and their associated explant procedures.

In one of its basic configurations, a prosthetic valve assembly comprising a prosthetic valve, a removable skirt, and at least one retaining suture. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise an annular frame movable between a radially compressed and a radially expanded configuration.

In some examples, the removable skirt is disposed radially outward to the frame.

In some examples, the removable skirt comprises one or more inflow loops extending from an inflow end of the removable skirt.

In some examples, the removable skirt one or more outflow loops extending from an outflow end of the removable skirt.

In some examples, the at least one retaining suture is coupled to the frame at a suture coupling end.

In some examples, the at least one retaining suture extends through the one or more inflow loops of the removable skirt, and through the one or more outflow loops of the removable skirt.

In some examples, the one or more inflow loops of the removable skirt and the one or more outflow loops of the removable skirt are arranged around the circumference of the frame.

In some examples, the removable skirt is configured to be released from the frame upon withdrawal of the at least one retaining suture from the one or more inflow loops of the removable skirt and from the one or more outflow loops of the removable skirt.

In one of its basic methods, a method of assembling a prosthetic valve assembly comprises attaching a first skirt to a frame of a prosthetic valve. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the method comprises releasably coupling a second skirt to the first skirt by passing at least one retaining suture through outflow loops of the first skirt and of the second skirt, and through inflow loops of the first skirt and of the second skirt.

In some examples, the method comprises attaching a suture coupling end of the at least one retaining suture to the frame.

In one of its basic methods, a method of explanting a prosthetic valve comprises accessing an implantation site of a prosthetic valve assembly. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the prosthetic valve assembly optionally comprises the prosthetic valve having a first skirt attached to a frame, and a second skirt releasably coupled to the first skirt by at least one retaining suture.

In some examples, the at least one retaining suture is optionally attached to the frame at a suture coupling end.

In some examples, the at least one retaining suture optionally extends through one or more inflow loops of the first and the second skirts, and through one or more outflow loops of the first and the second skirts.

In some examples, the method comprises cutting the at least one retaining suture by a cutting tool.

In some examples, the method optionally comprises grasping a loose end of the at least one retaining and pulling it so as to withdraw the at least one retaining suture from the one or more outflow loops of the first and the second skirts and from the one or more inflow loops of the first and the second skirts, thereby separating the prosthetic valve from the second skirt.

In some examples, the method optionally comprises removing the prosthetic valve from the implantation site.

In one of its basic configurations, a prosthetic valve kit comprises a prosthetic valve having a longitudinal axis and a cutting member. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise an annular frame movable between a radially compressed and a radially expanded configuration.

In some examples, the prosthetic valve can comprise an outer skirt disposed around an outer surface of the annular frame and coupled to the annular frame.

In some examples, the cutting member can comprise at least one elongated segment.

In some examples, the at least one elongated segment can extend along the annular frame from a proximal end portion of the elongated segment to a distal end portion of the elongated segment.

In some examples, the at least one elongated segment is optionally disposed radially outward to the annular frame.

In some examples, the cutting member is optionally electrically conductive.

In some examples, a prosthetic valve assembly can comprise the prosthetic valve kit and an electric current source configured to provide an electric current to the cutting member.

In one of its basic methods, a method of cutting an outer skirt of a prosthetic valve comprises providing an electric current to a cutting member. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the prosthetic valve has a longitudinal axis and optionally comprises an annular frame.

In some examples, the outer skirt is optionally disposed around an outer surface of the annular frame.

In some examples, the outer skirt is optionally coupled to the annular frame.

In some examples, the cutting member optionally comprises at least one elongated segment extending along the annular frame from a proximal end portion of the elongated segment to a distal end portion of the elongated segment.

In some examples, the at least one elongated segment is optionally disposed radially outward to the annular frame.

In some examples, the method comprises axially pulling the at least one cutting member.

In one of its basic configurations, a prosthetic valve comprises an annular frame, a first skirt coupled to the frame, and at least one skirt removal device. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the annular frame is optionally movable between a radially compressed and a radially expanded configuration.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve.

FIG. 1B is a perspective view of a frame of the prosthetic valve of FIG. 1A.

FIG. 2 shows an exemplary delivery assembly comprising a delivery apparatus carrying a prosthetic device.

FIG. 3A shows exemplary first and second skirts separated from each other, illustrated in flattened configurations of the skirts.

FIG. 3B shows the first and the second skirts of FIG. 3A disposed over each other.

FIG. 4 is a perspective view of an exemplary prosthetic valve assembly, comprising a base skirt and an outer skirt, without an inner skirt.

FIG. 5 is a schematic cross-sectional view of the prosthetic valve assembly of FIG. 4.

FIG. 6 is a schematic cross-sectional view of an exemplary prosthetic valve assembly, comprising an inner skirt, a base skirt, and an outer skirt.

FIG. 7 is a perspective view of an exemplary prosthetic valve assembly, comprising a retaining suture defining angularly oriented suture crossing sections.

FIG. 8 is a perspective view of an exemplary prosthetic valve assembly, comprising two retaining sutures.

FIG. 9 is a schematic cross-sectional view of an exemplary prosthetic valve assembly, comprising an inner skirt and an outer skirt, without a base skirt.

FIG. 10 is a schematic cross-sectional view showing a cutting tool approximated to a retaining suture of an exemplary prosthetic valve assembly.

FIG. 11A shows an exemplary cutting member with a circumferential loop.

FIG. 11B shows a cross-sectional view of a first portion of the cutting member of FIG. 11A.

FIG. 11C shows a cross-sectional view of a second portion of the cutting member of FIG. 11B.

FIG. 12A is a schematic cross-sectional view of an exemplary prosthetic valve kit comprising a cutting member disposed between the frame and the outer skirt of the prosthetic valve.

FIG. 12B is a schematic cross-sectional view showing a cutting member of the prosthetic valve kit of FIG. 12A used for separating the outer skirt from the frame.

FIG. 13 is a schematic cross-sectional view of an exemplary prosthetic valve kit comprising a cutting member disposed over the outer surface of the outer skirt.

FIG. 14 shows an exemplary cutting member with a plurality of elongated segments.

FIG. 15 shows an exemplary cutting member with an open circumferential segment.

FIG. 16 is a perspective view of an exemplary prosthetic valve kit having an elongated segment of a cutting members disposed between the frame and the outer skirt of the prosthetic valve.

FIG. 17 is a perspective view of an exemplary prosthetic valve kit having an elongated segment of a cutting members over the outer surface of the outer skirt.

FIG. 18A shows an exemplary cutting member with a loop segment comprising an electrically isolated portion.

FIG. 18B shows an exemplary cutting member with a loop segment comprising an electrically isolated face.

FIG. 19 is a perspective view of an exemplary prosthetic valve assembly, comprising single releasable skirt directly attached to the frame.

FIG. 20 is a perspective view of an exemplary prosthetic valve assembly, comprising single releasable skirt attached to eyelets of the frame.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

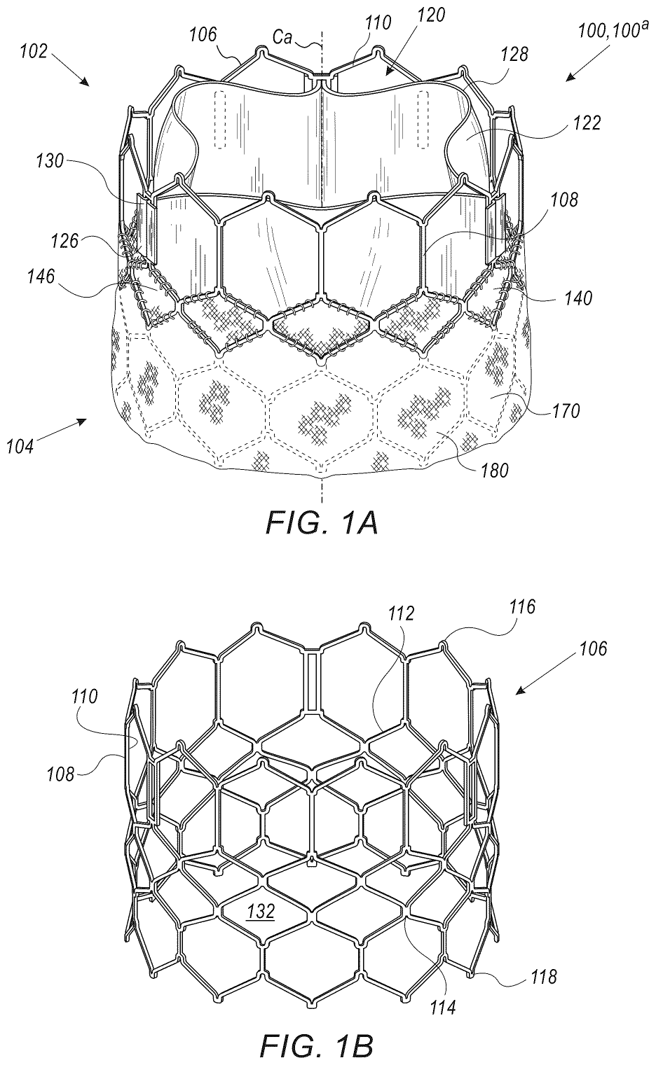

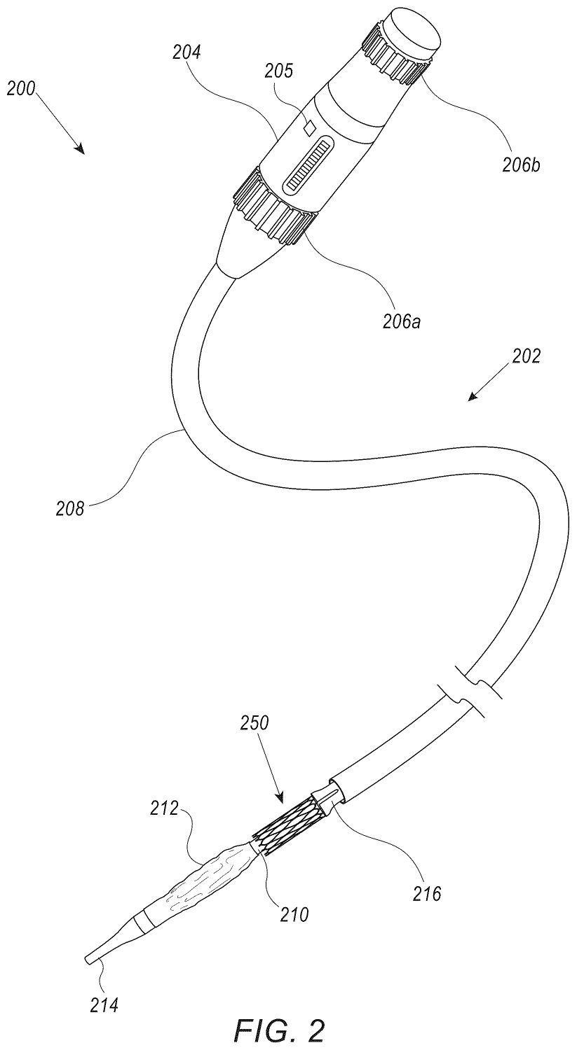

FIGS. 1A and 1B show perspective views of a prosthetic valve 100 with and without soft components attached thereto, according to some examples. FIG. 2 shows a perspective view of a delivery assembly 200, according to some examples. The delivery assembly 200 can include the prosthetic valve 100 and a delivery apparatus 202. The prosthetic valve 100 can optionally be on or releasably coupled to the delivery apparatus 202. The delivery apparatus can optionally include a handle 204 at a proximal end thereof, and a nosecone shaft (concealed from view in FIG. 2) extending distally from the handle 204, optionally having a nosecone 214 attached to its distal end.

The term “prosthetic valve”, as used herein, refers to any type of a prosthetic valve deliverable to a patient's target site over a catheter, which is radially expandable and compressible between a radially compressed, or crimped, state, and a radially expanded state. Thus, a prosthetic valve 100 can be crimped or retained by a delivery apparatus 202 in a compressed state during delivery, and then expanded to the expanded state once the prosthetic valve 100 reaches the implantation site. The expanded state may optionally include a range of diameters to which the valve may expand, between the compressed state and a maximal diameter reached at a fully expanded state. Thus, a plurality of partially expanded states may relate to any expansion diameter between radially compressed or crimped state, and maximally expanded state.

A prosthetic valve 100 of the current disclosure may include any prosthetic valve configured to be mounted within the native aortic valve, the native mitral valve, the native pulmonary valve, and the native tricuspid valve. While a delivery assembly 200 described in the current disclosure, includes a delivery apparatus 202 and a balloon expandable prosthetic device, such as prosthetic valve 100, it should be understood that the delivery apparatus 202 according to any example of the current disclosure can optionally be used for implantation of other prosthetic devices aside from prosthetic valves, such as stents or grafts.

A catheter deliverable prosthetic valve 100 can optionally be delivered to the site of implantation via the delivery assembly carrying the valve 100 in a radially compressed or crimped state, toward the target site, to be mounted against the native anatomy, by expanding the prosthetic valve 100 via various expansion mechanisms. Balloon expandable valves generally involve a procedure of inflating a balloon within a prosthetic valve, thereby expanding the prosthetic valve 100 within the desired implantation site. Once the valve is sufficiently expanded, the balloon is deflated and retrieved along with the delivery apparatus 202. Self-expandable valves include a frame that is shape-set to automatically expand as soon as an outer retaining capsule, which may be also defined as the distal portion of an outer shaft or the distal portion of a delivery shaft, is withdrawn proximally relative to the prosthetic valve.

Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209,904, each of which is incorporated herein by reference in its entirety), releasably coupled to respective actuation assemblies of the delivery apparatus, controlled via a handle for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The delivery assembly can be utilized, for example, to deliver a prosthetic aortic valve for mounting against the aortic annulus, to deliver a prosthetic mitral valve for mounting against the mitral annulus, or to deliver a prosthetic valve for mounting against any other native annulus.

FIGS. 1A-1B show an example of a prosthetic valve 100, which can optionally be a balloon expandable valve or any other type of valve, illustrated in an expanded state. The prosthetic valve 100 can comprise an outflow end 102, an inflow end 104, and a central longitudinal axis Ca extending in a direction from the inflow end 104 to the outflow end 102. In some instances, the outflow end 102 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The prosthetic valve 100 comprises an annular frame 106 movable between a radially compressed configuration and a radially expanded configuration, and a leaflet assembly 120 mounted within the frame 106. The frame 106 can be made of various suitable materials, including plastically-deformable materials such as, but not limited to, stainless steel, a nickel-based alloy (e.g., a cobalt-chromium or a nickel-cobalt-chromium alloy such as MP35N alloy), polymers, or combinations thereof. When constructed of a plastically-deformable materials, the frame 106 can be crimped to a radially compressed state on a balloon catheter 210 (shown in FIG. 2), and then expanded inside a patient by an inflatable balloon 212 or equivalent expansion mechanism. Alternatively or additionally, the frame 106 can be made of shape-memory materials such as, but not limited to, nickel-titanium alloy (e.g., Nitinol). When constructed of a shape-memory material, the frame 106 can be crimped to a radially compressed state and restrained in the compressed state by insertion into a shaft or equivalent mechanism of a delivery apparatus.

In the example illustrated in FIGS. 1A-1B, the frame 106 is an annular, stent-like structure comprising a plurality of intersecting struts 112. The frame 106 defines a frame inner surface 110 facing the central longitudinal axis Ca, and an opposite frame outer surface 108 facing away from the central longitudinal axis Ca. In the current disclosure, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 112 may be any elongated member or portion of the frame 106. The frame 106 can include a plurality of strut rungs that can collectively define one or more rows of cells 132. The frame 106 can have a cylindrical or substantially cylindrical shape having a constant diameter from the inflow end 104 to the outflow end 102 as shown, or the frame can vary in diameter along the height of the frame, as disclosed in U.S. Pat. No. 9,155,619, which is incorporated herein by reference.

The end portions of the struts 112 are forming apices 116 at the outflow end 102 and apices 118 at the inflow end 104. The struts 112 can intersect at additional junctions 114 formed between the outflow apices 116 and the inflow apices 118. The junctions 114 can be equally or unequally spaced apart from each other, and/or from the apices 116, 118, between the outflow end 102 and the inflow end 104.

At least some of the struts 112 can be pivotable or bendable relative to each other, so as to permit frame expansion or compression. For example, the frame 106 can be formed from a single piece of material, such as a metal tube, via various processes such as, but not limited to, laser cutting, electroforming, and/or physical vapor deposition, while retaining the ability to collapse/expand radially in the absence of hinges and the like.

A leaflet assembly 120 of the prosthetic valve 100 can optionally include a plurality of leaflets 122 (e.g., three leaflets), positioned at least partially within the frame 106, and configured to regulate flow of blood through the prosthetic valve 100 from the inflow end 104 to the outflow end 102. While three leaflets 122 arranged to collapse in a tricuspid arrangement, are shown in the example illustrated in FIG. 1A, it will be clear that a prosthetic valve 100 can include any other number of leaflets 122.

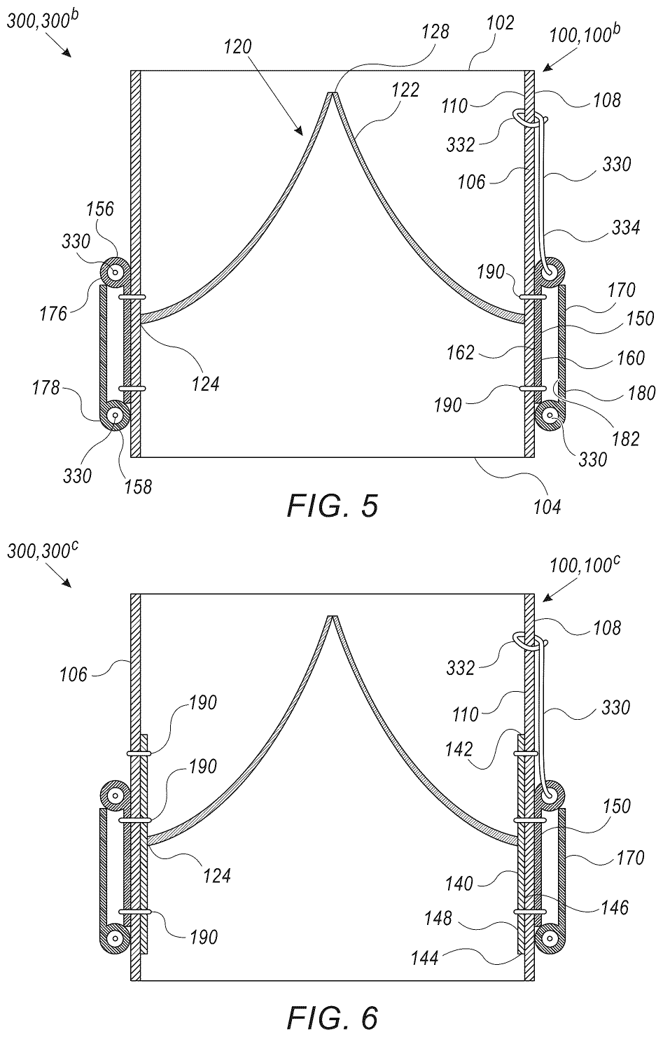

The inflow or cusp edges 124 (indicated, for example, in FIG. 5-6) of the leaflets 122 can optionally be secured to the frame 106 directly or indirectly, such as by being sutured directly to the frame, being sutured to an inner skirt, and/or via one or more connecting skirts. Further examples and methods of attaching skirts and seal members to a frame, as well as method and techniques for coupling leaflets 122 to the frame 106, with or without connecting skirts, are disclosed in US Pat. Publication No. 2018/0028310, which is incorporated herein by reference.

Adjacent leaflets 122 can optionally be arranged together to form commissures 130 that are coupled (directly or indirectly) to respective portions of the frame 106, thereby securing an upper portion (e.g., above the scalloped line) of the leaflet assembly 120 to the frame 106. In some examples, each leaflet 122 can optionally comprise opposing tabs 126. Each tab 126 can optionally be secured to an adjacent tab 126 of an adjacent leaflet 122 to form a commissure 130 that is secured to the frame 106. The tabs 126 can be folded in various manners, for example to form radially extending layers and circumferentially extending layers facing the frame. Radially extending layers can extend radially inward from a location on the frame 106 to free edge 128, also termed coaptation edges, of the leaflet.

During valve cycling, the leaflets 122 can articulate at the inner most edges of the tab layers, which helps space the leaflets away from the frame 106 during normal operation of the prosthetic valve. This is particular advantageous in cases where the prosthetic valve 100 is not fully expanded to its nominal size when implanted in a patient. As such, the prosthetic valve 100 can be implanted in a wider range of patient annulus sizes. Further details regarding transcatheter prosthetic valves, including the manner in which leaflets 122 can be coupled to the frame 106 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

According to some examples, the prosthetic valve 100 can optionally further comprise at least one skirt or sealing member. An inner skirt 140, extending can be secured to the inner surface 110 of the frame 106, configured to function, for example, as a sealing member to prevent or decrease perivalvular leakage. An inner skirt 140 can further function as an anchoring region for the leaflets 122 to the frame 106, and/or function to protect the leaflets 122 against damage which may be caused by contact with the frame 106, for example during valve crimping or during working cycles of the prosthetic valve 100. Additionally, or alternatively, the prosthetic valve 100 can optionally comprise an outer skirt 170 mounted on the outer surface 108 of the frame 106, configure to function, for example, as a sealing member retained between the frame 106 and the surrounding tissue of the native annulus against which the prosthetic valve is mounted, thereby reducing risk of paravalvular leakage (PVL) past the prosthetic valve 100.

The inner skirt 140 extends between an inner skirt inflow end 144 and an inner skirt outflow end 142, and defines an inner skirt outer surface 146, configured to face the frame inner surface 110 when coupled to frame 106, and an inner skirt inner surface 148 configured to face the central longitudinal axis Ca. The outer skirt 170 extends between an outer skirt inflow end 174 and an outer skirt outflow end 172, and defines an outer skirt inner surface 182, configured to face the frame outer surface 108 when coupled to frame 106, and an outer skirt outer surface 180 configured to face the surrounding anatomy when the valve 100 is implanted within a patient's body.

Any of the inner skirt 140 and/or outer skirt 170 can be made of various suitable biocompatible materials, such as, but not limited to, various synthetic materials (e.g., PET) or natural tissue (e.g. pericardial tissue). In some cases, the inner skirt 140 can optionally be formed of a single sheet of material that extends continuously around the inner surface 110 of frame 106. In some cases, the outer skirt 170 can optionally be formed of a single sheet of material that extends continuously around the outer surface 108 of frame 106.

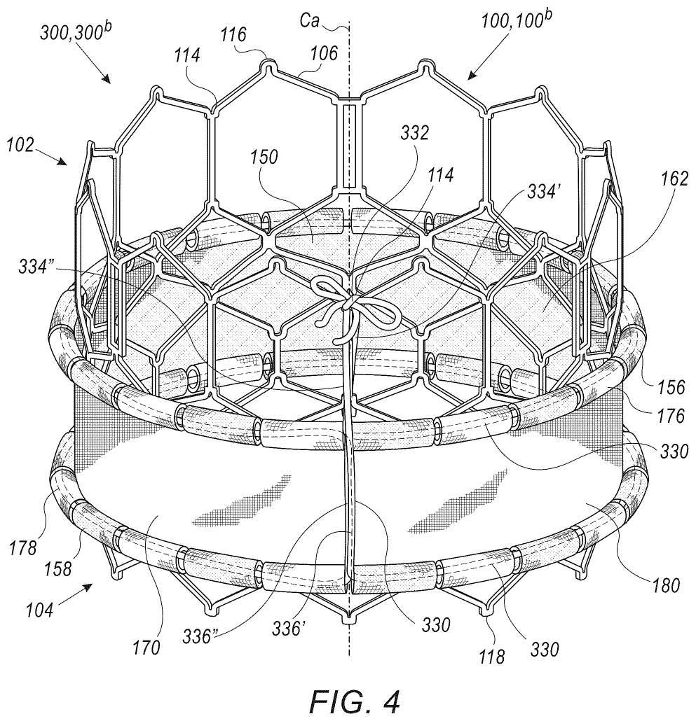

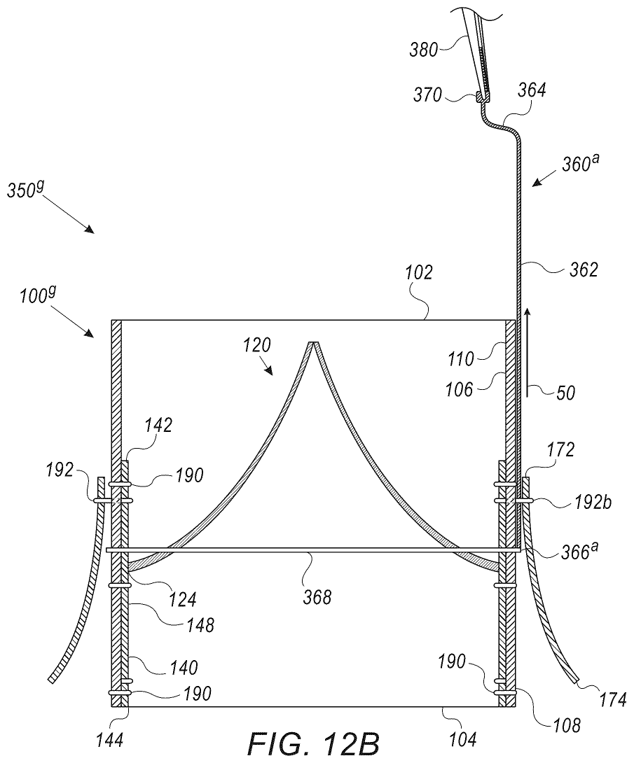

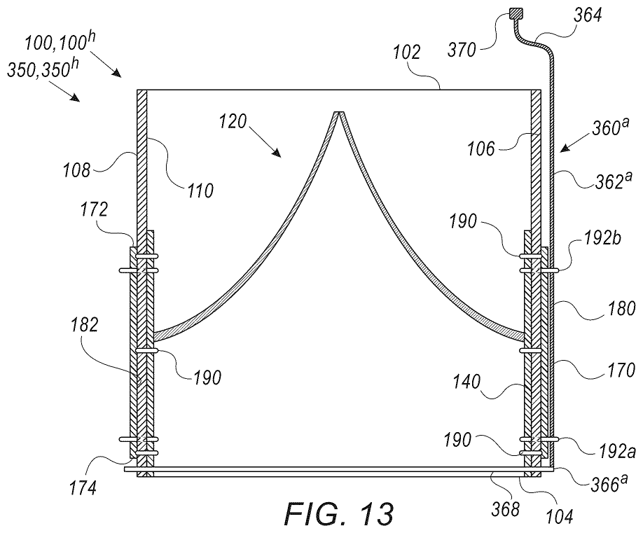

Various exemplary implementations for prosthetic valves 100, prosthetic valves assemblies 300 (indicated, for example, in FIGS. 4-10 and 19-20), prosthetic valve kits 350 (indicated, for example, in FIGS. 12A-15), and/or components thereof, can be referred to, throughout the specification, with superscripts, for ease of explanation of features that refer to such exemplary implementations. It is to be understood, however, that any reference to structural or functional features of any kit, assembly, device or component, without a superscript, refers to these features being commonly shared by all specific exemplary implementations that can be also indicated by superscripts. In contrast, features emphasized with respect to an exemplary implementation of any assembly, device or component, including prosthetic valve 100, prosthetic valve assembly 300, and/or prosthetic valve kit 350, referred to with a superscript, may be optionally shared by some but not necessarily all other exemplary implementations. For example, prosthetic valve 100ª illustrated in FIG. 1A is an exemplary implementation of prosthetic valve 100, and thus includes all of the features described for prosthetic valve 100 throughout the current disclosure, except that while a prosthetic valve 100 can be generally provided with any combination of skirts, prosthetic valve 100ª includes an inner skirt 140 that can be coupled to (e.g., sutured) and in contact with, the frame 106, and an outer skirt 170 that can be coupled to (e.g., sutured) and in contact with, the frame 106.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

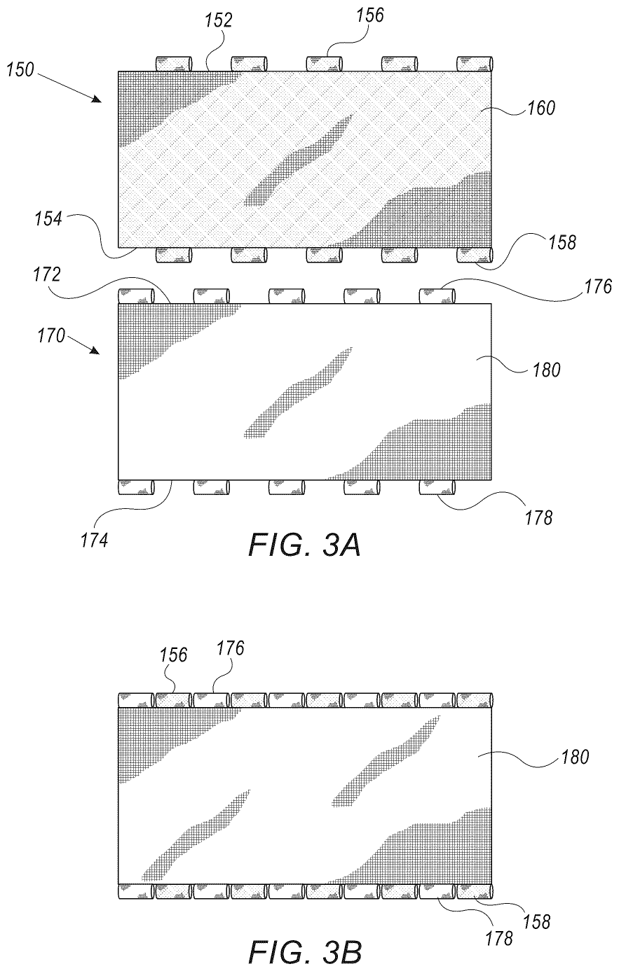

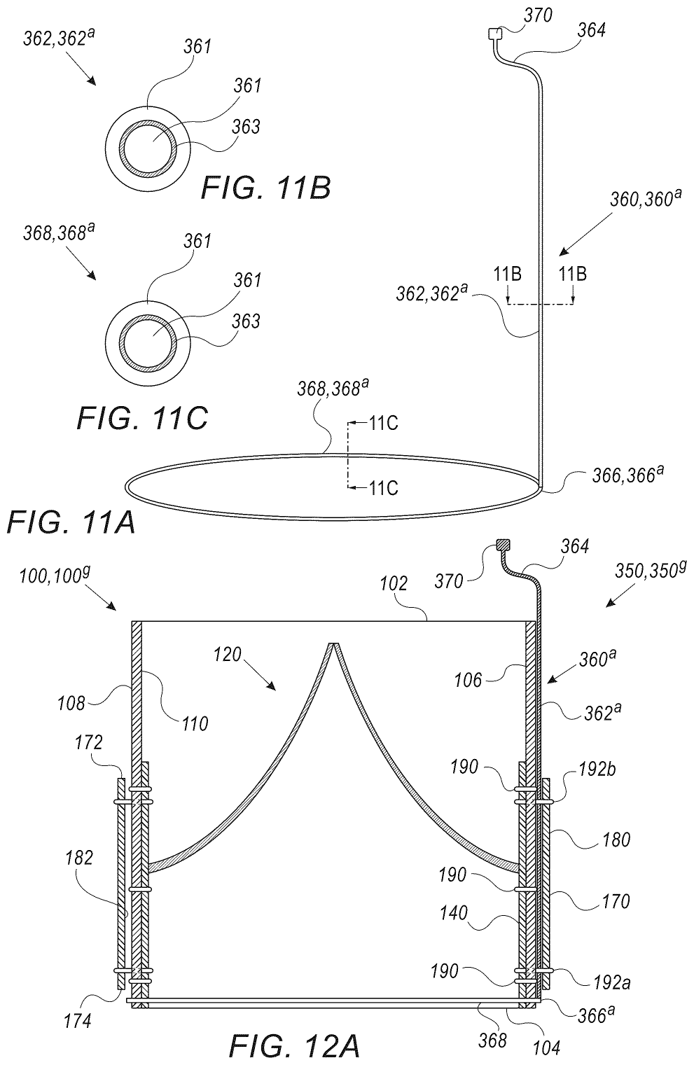

FIG. 2 illustrates a delivery assembly 200 that includes a delivery apparatus 202 adapted to deliver a prosthetic device 250, which can optionally be the prosthetic valve 100 described above with respect to FIGS. 1A-1B, a prosthetic valve assembly 300 according to examples that will be described below with respect to FIGS. 3-10, or a prosthetic valve kit 350 according to examples that will be described below with respect to FIGS. 11-15. According to some examples, the delivery apparatus 202 includes a handle 204 and at least one catheter extending therefrom, configured to carry a prosthetic device 250 (e.g., prosthetic valve 100, prosthetic valve assembly 300, or prosthetic valve kit 350) in a crimped state through the patient's vasculature. An exemplary delivery assembly 200 optionally comprises an exemplary delivery apparatus 202 configured to carry a balloon expandable prosthetic valve. The delivery apparatus 202 can optionally comprise a balloon catheter 210 having an inflatable balloon 212 mounted on its distal end. A balloon expandable prosthetic valve 100 or prosthetic valve assembly 300 can optionally be carried in a crimped state over the balloon catheter 210.

In some examples, a delivery apparatus 202 further comprises an outer delivery shaft 208. Optionally, an outer delivery shaft 208 of a delivery apparatus 202 can concentrically extend over the balloon catheter 210. In some examples, delivery apparatus 202 can optionally further comprise a push shaft 216 disposed over the balloon catheter 210, optionally between the balloon catheter 210 and the outer delivery shaft 208.

The outer delivery shaft 208, the push shaft 216, and the balloon catheter 210, can optionally be configured to be axially movable relative to each other. For example, a proximally oriented movement of the outer delivery shaft 208 relative to the balloon catheter 210, or a distally oriented movement of the balloon catheter 210 relative to the outer delivery shaft 208, can expose the prosthetic device 250 (e.g., prosthetic valve 100, prosthetic valve assembly 300, or prosthetic valve kit 350) from the outer delivery shaft 208.

A delivery apparatus 202 can optionally further include a nosecone 214 carried by a nosecone shaft. The nosecone shaft can optionally extend through a lumen of the balloon catheter 210.

The proximal ends of the balloon catheter 210, the outer delivery shaft 208, the push shaft 216, and/or the nosecone shaft, can optionally be coupled to the handle 204. During delivery of the prosthetic device 250, the handle 204 can be maneuvered by an operator (e.g., a clinician or a surgeon) to axially advance or retract components of the delivery apparatus 202, such as the nosecone shaft, the outer delivery shaft 208, the balloon catheter 210 and/or the push shaft 216, through the patient's vasculature and/or along the target site of implantation, as well as to inflate the balloon 212 mounted on the balloon catheter 210, so as to expand the prosthetic valve 100, and to deflate the balloon 212 and retract the delivery apparatus 202 once the prosthetic device 250 is mounted in the implantation site.

The handle 204 can optionally include a steering mechanism configured to adjust the curvature of the distal end portion of the delivery apparatus 202. In the illustrated example, the handle 204 includes an adjustment member, such as the illustrated rotatable knob 206a, which in turn is operatively coupled to the proximal end portion of a pull wire (not shown). The pull wire can extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the push shaft 216 relative to the balloon catheter 210. The handle can include additional knobs to control additional components of the delivery apparatus 202, such as positioning members that will be described in greater detail below.

The prosthetic device 250 (e.g., prosthetic valve 100, prosthetic valve assembly 300, or prosthetic valve kit 350) can be carried by the delivery apparatus 202 during delivery in a crimped state, and expanded, for example by balloon inflation, to secure it in a native heart valve annulus (such as an aortic annulus) or against a previously implanted prosthetic valve (for example, during valve-in-valve implantation procedures). The prosthetic device 250 can optionally be initially crimped over the balloon catheter 210, proximal to the balloon 212. Because prosthetic device 250 is crimped at a location different from the location of balloon 212, prosthetic device 250 can be crimped to a lower profile than would be possible if it was crimped on top of balloon 212. This lower profile permits the clinician to more easily navigate the delivery assembly 200 (including crimped prosthetic device 250) through a patient's vasculature to the treatment location. The lower profile of the crimped prosthetic valve is particularly helpful when navigating through portions of the patient's vasculature which are particularly narrow, such as the iliac artery.

The balloon 212 can optionally be secured to balloon catheter 210 at the balloon's proximal end, and to either the balloon catheter 210, the nosecone shaft, or the nosecone 214 at its distal end. The distal end portion of the push shaft 216 is positioned proximal to the outflow end 102 of prosthetic device 250.

When reaching the site of implantation, the deflated balloon 212, carrying crimped prosthetic device 250 thereover, can be advanced to the target site to expand the prosthetic valve. Prior to balloon 212 inflation, the push shaft 216 is optionally advanced distally, allowing its distal end portion to contact and push against the outflow end 102 of prosthetic valve 100, pushing the valve 100 distally therewith. The distal end of push shaft 216 is dimensioned to engage with the outflow end 102 of prosthetic valve 100 in a crimped configuration of the valve. In some examples, the distal end portion of the push shaft 216 can optionally be flared radially outward, to terminate at a wider-diameter that can contact the prosthetic valve 100 in its crimped state. Optionally, push shaft 216 can then be advanced distally, pushing the prosthetic valve 100 therewith, until the crimped prosthetic valve 100 is disposed around the balloon 212, at which point the balloon 212 can be inflated to radially expand the prosthetic valve 100. Once the prosthetic valve 100 is expanded to its functional diameter within a native annulus or within a previously implanted prosthetic valve, the balloon 212 can be deflated, and the delivery apparatus 202 can be retrieved from the patient's body.

In some examples, any delivery assembly of the current disclosure can optionally be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

As discussed above, after a period of use of an implanted prosthetic valve, it may be desirable or necessary to explant the prosthetic valve so that a new prosthetic valve can be implanted. Such explant procedures can be complicated for a surgeon to perform and/or traumatic to the patient depending on the anatomy of the patient. For example, overtime soft portions of the prosthetic valve, such as an outer skirt, may experience tissue ingrowth. During explant, the ingrown tissue may make it difficult for the prosthetic valve to be removed from the implantation site, which may thereby make the procedure more complex for the surgeon to perform, may increase the time required to perform the surgical procedure, and/or may require significant cutting or damage to the surrounding tissue (or other tissues) as the surgeon attempts to remove the prosthetic valve structure from the patient.

Accordingly, a prosthetic valve or assembly that can facilitate explant procedures is desired. For example, there is a need for prosthetic valves or assemblies and explant procedures that limit tissue trauma and improve the ease and/or expedite of the removal process.

Described herein are examples of prosthetic valves that include skirt removal devices, configured to facilitate detachment of an outer skirt from the remainder of the valve to allow for easier removal of valve from the surrounding anatomy in explantation procedure. Skirt removal devices disclosed herein can optionally include retaining sutures 330 or cutting members 360, as will be described in further detail below. Skirt removal devices in the form of one or more retaining sutures are disclosed herein as part of prosthetic valve assemblies 300 (shown, for example, in FIGS. 4-10) that include an annular frame, a plurality of leaflets (that is, a leaflet assembly) attached to the frame, a removable outer skirt, and optionally another skirt that can be an inner skirt or a base skirt. The removable outer skirt can optionally be attached or secured to another component of the prosthetic valve or prosthetic valve assembly, such as the frame or another skirt, in a detachable manner that allows the rest of the prosthetic valve, including the frame, to be easily separated or released from the skirt, for example, prior to an implant procedure of a prosthetic valve or during explant of a prosthetic valve.

In some examples, the leaflets can optionally be attached to the frame via commissures formed by joining pairs of adjacent tabs of the leaflets. The cusp edges of the leaflets can optionally be attached to a skirt such as the outer skirt, an inner skirt mounted to an inner surface of the frame, a base skirt mounted between the frame and the outer skirt, or directly to selected struts of the frame. Optionally, the frame can be radially expandable and compressible, and can be configured for transcatheter implantation.

In some examples, a prosthetic valve assembly comprises a prosthetic valve that can optionally include a first skirt attached to the frame, and a second skirt releasably coupled to the first skirt, configured to be detached from the first skirt, and therefore from the remainder of the prosthetic valve, for example if explantation procedure is required. The second skirt can optionally be a removable outer skirt 170, having an outer skirt outer surface 180 that can optionally be exposed to the surrounding anatomy when the prosthetic valve is implanted in a patient's body. The terms “outer skirt 170” and “removable outer skirt 170”, as used herein, are interchangeable. The prosthetic valve comprises the frame, and as mentioned above, can optionally comprise a first skirt. The first skirt can optionally be coupled (e.g., sutured) to the frame, while the second skirt or outer skirt can optionally be coupled by one or more retaining sutures to the first skirt.

In some examples, the first skirt is a base skirt disposed between the frame and the second skirt. In such a case, both the first skirt and the second skirt are disposed around the outer side or outer surface of the frame, with an inner surface of the first skirt or base skirt in direct contact with the frame, and with the second skirt or outer skirt having an inner surface in direct contact with the outer surface of the base skirt. Such a prosthetic valve assembly can be provided without an additional inner skirt, or can optionally further include an inner skirt having an outer surface in direct contact with the frame inner surface. In some examples, the first skirt is the inner skirt, and the second skirt is the outer skirt. In such examples, the prosthetic valve assembly can optionally be provided without a base skirt.

FIG. 3A shows exemplary first and second skirts separated from each other, illustrated in flattened configurations of the skirts. FIG. 3B shows the first and the second skirts disposed over each other, illustrated in a flattened configuration of the skirts. In the example illustrated in FIGS. 3A-3B, the first skirt is a base skirt 150, and the second skirt is the outer skirt 170. The base skirt 150 extends between a base skirt inflow end 154 and a base skirt outflow end 152, and defines a base skirt inner surface 162, configured to face and optionally contact the frame outer surface 108 when coupled to frame 106, and a base skirt outer surface 160 configured to face and optionally contact the outer skirt inner surface 182 when both skirts are assembled together as shown in FIG. 3B. Similar to inner skirt and outer skirt described above, the base skirt 150 can be made of various suitable biocompatible materials, such as, but not limited to, various synthetic materials (e.g., PET) or natural tissue (e.g. pericardial tissue). In some cases, the base skirt 150 can optionally be formed of a single sheet of material that extends continuously around the outer surface 108 of frame 106.

Each of the first and the second skirts, such as the base skirt 150 and the outer skirt 170, includes a plurality of loops, such as one or more inflow loops extending from each skirt's inflow end, and one or more outflow loops extending from each skirt's outflow end. The inflow loops and the outflow loops of both skirts are circumferentially offset from each other when the skirts are assembled together, as shown in FIG. 3B.

In some examples, the base skirt 150 comprises a plurality of base skirt outflow loops 156 extending from the base skirt outflow end 152, and a plurality of base skirt inflow loops 158 extending from the base skirt inflow end 154. In some examples, the number of base skirt outflow loops 156 can optionally match the number of base skirt inflow loops 158. In some examples, the of base skirt outflow loops 156 can optionally be circumferentially aligned with the base skirt inflow loops 158.

In some examples, the outer skirt 170 comprises a plurality of outer skirt outflow loops 176 extending from the outer skirt outflow end 172, and a plurality of outer skirt inflow loops 178 extending from the outer skirt inflow end 174. In some examples, the number of outer skirt outflow loops 176 can optionally match the number of outer skirt inflow loops 178. In some examples, the of outer skirt outflow loops 176 can optionally be circumferentially aligned with the outer skirt inflow loops 178. In some examples, the base skirt 150 and the outer skirt 170 are similarly dimensioned, such as by having similar heights between their inflow and outflow ends. The term “circumferentially aligned”, as used herein with respect to skirt loops, such as either inflow loops or outflow loops of the first and the second skirts, refer to the inflow loops of the first skirt being at the same axial position (along central longitudinal axis Ca or an axis parallel thereto) as the inflow loops of the second skirt, and/or to the outflow loops of the first skirt being at the same axial position as the outflow loops of the second skirt, such that the respective inflow loops and/or outflow loops of both skirts are arranged next to each other around the circumference of the frame.

The first 150 and second 170 skirts can optionally be arranged around the frame 106 as shown in FIG. 4, to form prosthetic valve assembly 300. A prosthetic valve assembly 300 of the type shown in FIG. 4, for example, includes a first skirt (e.g., base skirt 150) attached to the frame 106, such as by sutures or any other couplers, and a second skirt (i.e., outer skirt 170) releasably attached to the first skirt by at least one retaining suture 330 extending through skirt loops of the first and the second skirt, wherein the retaining suture 330 is coupled to the frame 106, such as by a suture coupling end 332 that can include a knot or any other suitable manner of attachment.

As shown in FIG. 3B, the base skirt outflow loops 156 and the outer skirt outflow loops 176 can optionally be arranged in an alternating manner along the circumferential direction when the skirts are coupled to the frame 106. The base skirt inflow loops 158 and the outer skirt inflow loops 178 can optionally be similarly arranged in an alternating manner along the circumferential direction.

FIGS. 4 and 5 show a view in perspective and a sectional side view, respectively, of an exemplary prosthetic valve assembly 300b. Prosthetic valve assembly 300b is an exemplary implementation of prosthetic valve assembly 300, and thus includes all of the features described for prosthetic valve assembly 300 throughout the current disclosure, except that the prosthetic valve 100b of prosthetic valve assembly 300b comprises a base skirt 150 serving as the first skirt of prosthetic valve assembly 300h, the second skirt of prosthetic valve assembly 300b is the outer skirt 170, and the prosthetic valve 100h is shown to be devoid of an inner skirt. Prosthetic valve assembly 300b is also shown to include a single retaining suture 330 extending through some of the loops of both skirts, and in some examples, through all of the loops of both skirts.

As shown, the base skirt 150 is disposed around the frame 106, such that the base skirt inner surface is facing and optionally contacting the frame outer surface 108. The outer skirt 170 is disposed around the base skirt 150, such that the outer skirt inner surface 182 is facing and optionally contacting the base skirt outer surface 160. The base skirt 150 and the outer skirt 170 can optionally be aligned with each other, such that the base skirt inflow end 154 and/or base skirt inflow loops 158 are axially aligned with the outer skirt inflow end 174 and/or outer skirt inflow loops 178, and the base skirt outflow end 152 and/or base skirt outflow loops 156 are axially aligned with the outer skirt outflow end 172 and/or outer skirt outflow loops 176, respectively. The term “axially aligned”, as used herein with respect to inflow and/or outflow ends of the skirts, refers to such ends being at the same axial position along the central longitudinal axis Ca (or an axis parallel thereto).

The base skirt 150 can optionally be attached to the frame 106 in a conventional manner, such as by sutures 190 (see FIG. 5) or other couplers, not necessarily configured to be releasably detached from the frame 106. The outer skirt 170 is releasably coupled to the base skirt 150 by a retaining suture 330. A retaining suture 330 is coupled to the frame 106 at a suture coupling end 332. In some examples, the suture coupling end 332 is a knot by which the retaining suture 330 is tied to the frame 106. The suture coupling end 332 (e.g., a knot) can optionally be axially offset from any of the base skirt 150 and outer skirt 170. In the illustrated example, the suture coupling end 332 is formed between the outflow ends 152, 172 of the skirt 150, 170 and the outflow end 102 of the frame 106. It is to be understood that the outer skirt 170 is shown as being spaced away from the base skirt 150 in cross-sectional views, such as FIG. 5, for illustrative purpose only, and that the outer skirt 170 may be regularly laid over the base skirt 150 such that the outer skirt inner surface 182 may optionally be in direct contact with the base skirt outer surface 160.

The retaining suture 330 extends from the suture coupling end 332 towards the outflow loops 156 and 176 of the base skirt 150 and the outer skirt 170, respectively, defining a suture offset section 334 between the suture coupling end 332 and the loops 156, 176, indicated as distally extending suture offset section 334′ in FIG. 4. The retaining suture 330 is then passed through the base skirt outflow loops 156 and outer skirt outflow loops 176 along the circumference of the prosthetic valve 100, and when it exits from the final outflow loop, the retaining suture 330 extends towards the inflow loops 158 and 178 of the base skirt 150 and the outer skirt 170, respectively, defining a suture crossing section 336 passed over the outer skirt outer surface 180 between the outflow loops 156, 176 and the inflow loops 158, 178, indicated as distally extending suture crossing section 336′ in FIG. 4.

The retaining suture 330 is then passed through the base skirt inflow loops 158 and outer skirt inflow loops 178 along the circumference of the prosthetic valve 100, and when it exits from the final inflow loop, it extends proximally towards the suture coupling end 332, defining an additional suture crossing section 336 and suture offset section 334 continuous therewith, indicated as proximally extending suture crossing section 336″ and suture offset section 334″, which can optionally be substantially parallel to the distally extending suture crossing section 336′ and suture offset section 334′.

During assembly of the skirts 150, 170 to frame 106, a knot or any other coupler of a suture coupling end 332 can optionally be formed at the final stage, after passing the retaining suture 330 along the path described above, by tying the ends of suture offset sections 334′ and 334″ together around one or more struts 112 and/or junctions 114 of the frame 106. It is to be understood that attachment of the retaining suture 330 to the frame 106 at the suture coupling end 332 is not necessarily performed by tying the suture around the frame to form a knot. In some examples, the suture coupling end 332 can optionally be attached to a portion of the prosthetic valve via an adhesive. In some examples, the suture coupling end 332 can optionally be secured to a strut 112 by being at least partially disposed beneath a suture or an elastomeric member covering the strut. Thus, it is to be understood that any reference of the suture coupling end 332 as a knot in the current disclosure is not meant to be limiting, and the suture coupling end 332 may be any alternative appropriate attachment means.

When the suture coupling end 332 is offset from the skirts 150, 170, the suture offset sections 334 can optionally be exposed portions of the retaining suture 330, above (i.e., proximal to) the skirts 150, 170, to allow easy access of the surgeon to these portions when detachment of the outer skirt is required. For example, a surgeon can easily access the suture coupling end 332 and/or suture offset sections 334 with a cutting tool (such as a blade, scalpel, scissors, and the like) and cut the retaining suture 330, or with a grabber (not shown) configured to grab the suture coupling end and pull it in a manner that can unravel a knot of release any other form by which the suture coupling end 332 is attached. When the retaining suture 330 is cut or otherwise released (including, for example, unraveling a knot thereof by a grabber), the loose end can be grasped and pulled by the surgeon, to withdraw the entire retaining suture 330 from the loops 156, 176, 158, 178. Once the retaining suture 330 is removed, the outer skirt 170 is separated from the base skirt 150 and the remainder of the prosthetic valve 100. This can allow removal of the prosthetic valve 100 with the first skirt (e.g., base skirt 150), from the site of implantation, while the outer skirt 170, that may have attached to the surrounding anatomy due to tissue ingrowth thereover, can remain in place, and a replacement prosthetic valve can be implanted therein.

FIG. 6 shows a sectional side view of an exemplary prosthetic valve assembly 300^{c}, that can be identical to any of the examples described above with respect to prosthetic valve assembly 300^{b}, except that the prosthetic valve 100^{c} of prosthetic valve assembly 300^{c} further comprises an inner skirt 140 disposed around the frame inner surface 110, in addition to the base skirt 150 and the outer skirt 170. Thus, prosthetic valve assembly 300^{c} can include a total of three skirts. In some examples, the base skirt 150 can optionally be sutured to the inner skirt 140, as shown in FIG. 6. In some examples, each of the base skirt 150 and the inner skirt 140 can optionally be separately coupled to the frame 106, such as by different sets of sutures 190.

Separation of the prosthetic valve 100^{c} from the outer skirt 170 of prosthetic valve assembly 300^{c} can optionally be performed in the same manner described above, in which case, both the base skirt 150 and the inner skirt 140 will remain attached to the frame 106 after separation, leaving only the outer skirt 170 optionally attached to the surrounding anatomy during removal of the prosthetic valve 100^{c} from the implantation site.

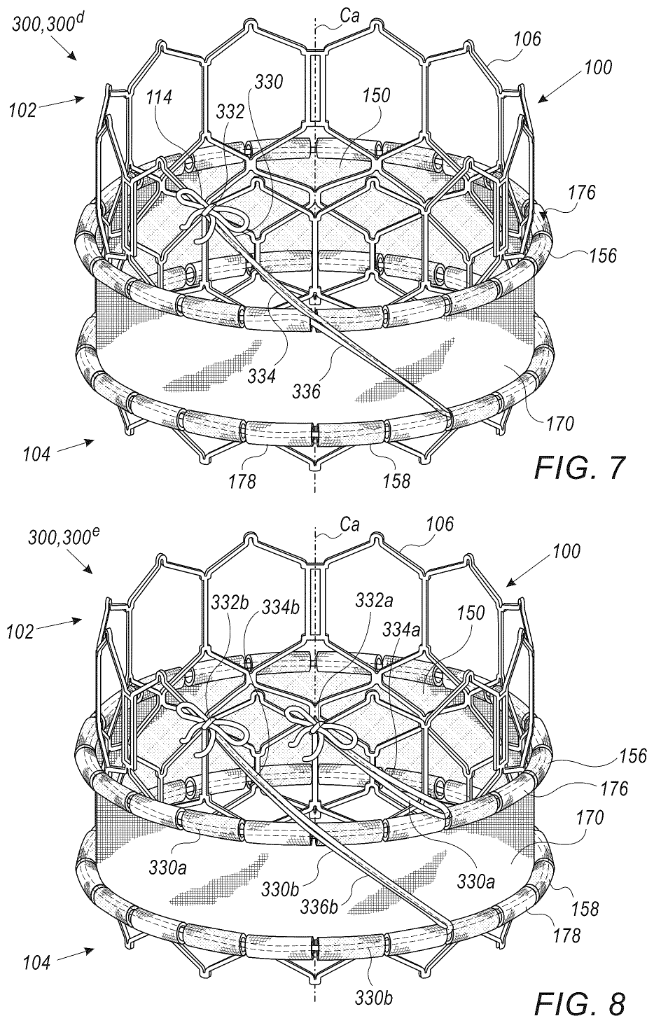

FIG. 7 shows a view in perspective of an exemplary prosthetic valve assembly 300^{d}. Prosthetic valve assembly 300^{d} is an exemplary implementation of prosthetic valve assembly 300, and thus includes all of the features described for prosthetic valve assembly 300 throughout the current disclosure, except that the suture crossing sections 334 of prosthetic valve assembly 300^{d} extend angularly relative to the vertical direction of central longitudinal axis Ca. This can be in contrast to the configuration illustrated for prosthetic valve assembly 300^{b} in FIG. 4, for example, showing the suture crossing sections 334 extending substantially vertically, parallel to the central longitudinal axis Ca, between the inflow ends 154, 174 and outflow ends 152, 172 of base and outer skirts 150, 170, respectively.

When the retaining suture 330 is cut and pulled to release it from the loops of the skirts, the vertical direction of the suture crossing sections 334 illustrated in FIG. 4 may introduce some resistance to the pull force. In contrast, the angular orientation of the suture crossing sections 334 illustrated in FIG. 7 allows the retaining suture 330 to be easily pulled away and released from the skirts 150, 170.

In some examples, the angular orientation of the suture crossing sections 334 relative to the longitudinal axis Ca is between 30° and 60°. In some examples, the angular orientation of the suture crossing sections 334 relative to the longitudinal axis Ca (or an axis parallel thereto) is between 40° and 50°. In some examples, the angular orientation of the suture crossing sections 334 relative to the longitudinal axis Ca is about 45°. In some examples, the suture crossing sections 336 are similarly angled between the skirts 150, 170 and the suture coupling end 332. The suture crossing sections 336 can optionally be continuous with the suture crossing sections 334, thus having the same angular orientation with respect to the longitudinal axis Ca.

It is to be understood that prosthetic valve assembly 300^{d} is shown in FIG. 7 to include a base skirt 150 and an outer skirt 170, without an inner skirt, by way of illustration and not limitation, and that angular orientation of the suture crossing sections 334 can optionally be implemented for any other exemplary prosthetic valve assembly 300 disclosed herein, including, for example, prosthetic valve assembly 300^{c} that includes an inner skirt 140 in addition to the base skirt 150 and the outer skirt 170.

FIG. 8 shows a view in perspective of an exemplary prosthetic valve assembly 300^{e}. Prosthetic valve assembly 300^{e} is an exemplary implementation of prosthetic valve assembly 300, and thus includes all of the features described for prosthetic valve assembly 300 throughout the current disclosure, except that prosthetic valve assembly 300^{e} includes two retaining sutures 330, namely a first retaining suture 330a that can be coupled to the frame 106 at a first suture coupling end 332a, and a second retaining suture 330b that can be coupled to the frame 106 at a second suture coupling end 332b.

First retaining suture 330a extends along a suture offset section 334a from the first suture coupling end 332a towards the outflow loops 156 and 176, passed through the outflow loops 156 and 176, and then extends back along another suture offset section 334a back toward the first suture coupling end 332a. As shown, since the first retaining suture 330a does not extend toward and does not pass through the inflow loops 158 and 178, it does not include suture crossing sections.

Second retaining suture 330b extends along a suture offset section 334b and suture crossing section 336b from the second suture coupling end 332b towards the inflow loops 158 and 178, passed through the inflow loops 158 and 178, and then extends back along another suture crossing section 336b and suture offset section 334b back toward the second suture coupling end 332b.

It is to be understood that prosthetic valve assembly 300^{c} is shown in FIG. 8 to include a base skirt 150 and an outer skirt 170, without an inner skirt, by way of illustration and not limitation, and that retaining sutures 330 can optionally be implemented for any other exemplary prosthetic valve assembly 300 disclosed herein, including, for example, prosthetic valve assembly 300^{c} that includes an inner skirt 140 in addition to the base skirt 150 and the outer skirt 170.

While both retaining sutures 330 are shown in FIG. 8 to be angularly oriented relative to longitudinal axis Ca, in a similar manner to that described above for prosthetic valve assembly 300^{d} and illustrated in FIG. 7, the orientation of retaining sutures 330 is not limited, and they can be vertically oriented as illustrated in FIG. 4, or assume any other orientation. Likewise, while the suture offset sections 334a are shown to be parallel to the suture offset sections 334b and suture crossing sections 336b, it is to be understood that each can be oriented in a different direction. The suture coupling ends 332a and 332b can optionally be spaced apart from each other as illustrated, or can optionally be attached to the same region of frame 106.

In order to separate the outer skirt 170 from the base skirt 150 and the remainder of prosthetic valve 100, each of the retaining sutures 330a and 330b needs to be cut. Pulling on a loose end of the first retaining suture 330a will release the suture 330a from the outflow loops 156 and 176, thereby separating the outer skirt outflow end 172 from the base skirt outflow end 152. Similarly, pulling on a loose end of the second retaining suture 330b will release the suture 330b from the inflow loops 158 and 178, thereby separating the outer skirt inflow end 174 from the base skirt inflow end 154. The sutures 330a and 330b can be simultaneously pulled, or sequentially pulled one after the other (in any order). While cutting and pulling on two sutures is required for full separation between the outer skirt 170 and the base skirt 150 in this configuration, each of the sutures can advantageously be pulled in an easier manner due to the limited path it extends along.

While a single retaining suture 330 is described and illustrated with respect to FIGS. 4-7, and two retaining sutures 330 are described and illustrated with respect to FIG. 8, any other number of retaining sutures 330 can be used, including more than two retaining sutures 330.

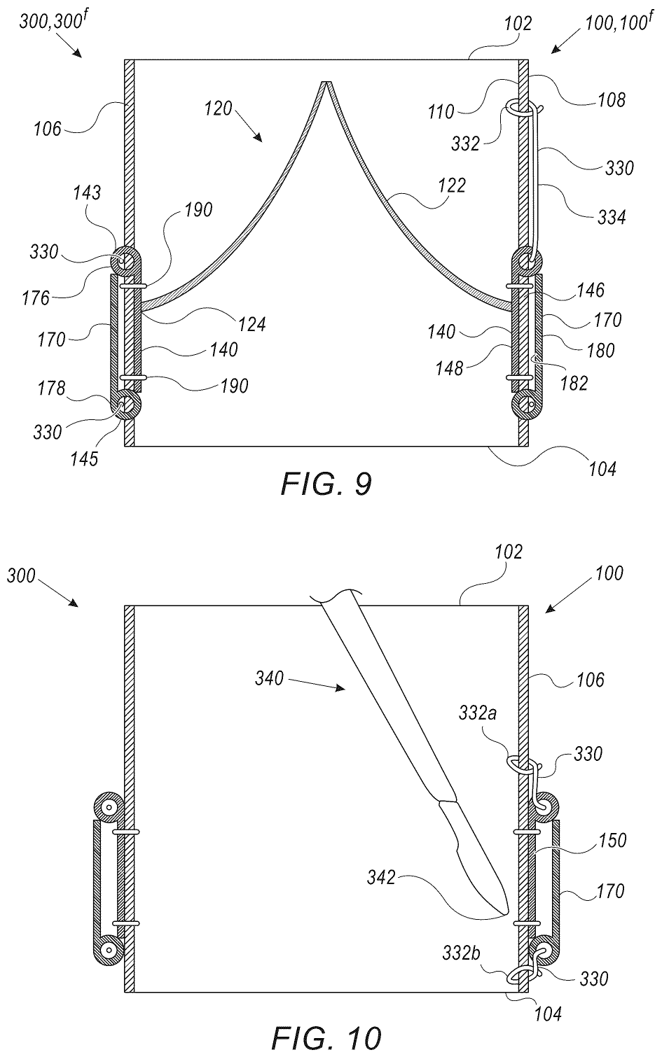

FIG. 9 shows a sectional side view of an exemplary prosthetic valve assembly 300^{f}. Prosthetic valve assembly 300^{f} is an exemplary implementation of prosthetic valve assembly 300, and thus includes all of the features described for prosthetic valve assembly 300 throughout the current disclosure, except that the prosthetic valve 100 of prosthetic valve assembly 300^{f} comprises an inner skirt 150 serving as the first skirt of prosthetic valve assembly 300^{f}, an is devoid of a base skirt disposed around an outer surface 108 of the frame 106. The inner skirt 140 in such examples comprises a plurality of inner skirt outflow loops 143 extending from the inner skirt outflow end 142, and a plurality of inner skirt inflow loops 145 extending from the inner skirt inflow end 144.

It is to be understood that releasably coupling the outer skirt 170 to the inner skirt 140 in such examples, can optionally be implemented according to any of the examples described for coupling the outer skirt 170 to the base skirt 150 with respect to FIGS. 3A-8 above, mutatis mutandis. Any examples described above with respect to base skirt 150, base skirt outflow end 152 and outflow loops 156 extending therefrom, base skirt inflow end 154 and inflow loops 158 extending therefrom, base skirt outer surface 160, and base skirt inner surface 162, can optionally be similarly implemented by being replaced with the corresponding inner skirt 140, inner skirt outflow end 142 and outflow loops 143 extending therefrom, inner skirt inflow end 144 and inflow loops 145 extending therefrom, inner skirt outer surface 146, and inner skirt inner surface 148.

For example, outer skirt 170 can optionally be coupled to inner skirt 140 by a single retaining suture 330 as described above with respect to FIG. 4, or by a first retaining suture 330a and a second retaining suture 330b as described above with respect to FIG. 8. Similarly, retaining sutures 330 can optionally form vertically-oriented suture offset sections 334 and/or suture crossing sections 336 as described above with respect to FIG. 4, or form angularly-oriented suture offset sections 334 and/or suture crossing sections 336 as described above with respect to FIGS. 7-8.

In some examples, the outer surface of the second skirt, such as the outer surface 180 of outer skirt 170, is configured to encourage tissue ingrowth, to help seal and anchor the prosthetic valve 100 against the native annulus. In some examples, the outer skirt 170 can include polyethylene terephthalate (PET) or another form of material that is configured to allow tissue ingrowth. In some examples, the material of the outer skirt 170 may be fabricated to allow tissue ingrowth. For example, a knit pattern of the material of the outer skirt 170 may be configured to allow tissue ingrowth, with a large knit pattern or other forms of knit patterns. A knit PET fabric may be utilized in some examples. In some examples, the outer skirt 170 may include yarns (e.g., textured yarns) extending radially outward.

In some examples, the outer skirt outer surface 180 comprises a pile or plush layer of fabric (for example, formed from PET yarns), such as a velour, terrycloth, or a towel. In other words, the fabric used to form the outer skirt outer surface 180 can include large number of filaments, such as closed loops (a looped pile), open fringes (a cut pile) or other filament forms, which promote tissue ingrowth into the filaments and sealing of the outer skirt via. The pile layer of the fabric can enhance sealing by mechanically slowing the flow of blood by creating a large micro-scale face area. In such examples, the pile layer can agitate the surrounding blood and/or tissue by exposure thereof to a large surface area, which can stimulate the foreign body response. Further details of skirts that are configured to promote tissue ingrowth are disclosed in U.S. Pat. No. 11,13,600 and U.S. Patent Publication Nos. 2019/0365530 and 2019/0374337, each of which is incorporated by reference herein.

In some examples, such as when the outer skirt outer surface 180 is configured to encourage tissue ingrowth, the outer skirt inner surface 182 and/or the outer surface of the first skirt, which can be the base skirt 150 and/or inner skirt 140, can optionally be configured to be relatively thromboresistant. In some examples, the base skirt 150 and/or inner skirt 140 can optionally include at least one or more of a polytetrafluoroethylene (PTFE), an ultra-high molecular weight polyethylene (UHMWPE), or a coated thermoplastic polyurethane (TPU). The polytetrafluoroethylene (PTFE) may comprise expanded polytetrafluoroethylene (ePTFE) in some examples as desired. Various other thromboresistant materials may be utilized as desired.

In some examples, a thromboresistant coating may be utilized with the inner surface 182 of the outer skirt 170, or surface of the base skirt 150 and/or inner skirt 140. For example, a coating of PTFE or ePTFE may be applied to any of the outer skirt inner surface 182, base skirt outer surface 160, and/or inner skirt outer surface 146. In some examples, any of the outer skirt inner surface 182, base skirt outer surface 160, and/or inner skirt outer surface 146, can optionally be coated in a thromboresistant material such as a hydrogel coating, a heparin coating, a silicone coating, or other materials or combinations thereof.

In some examples, the material of the base skirt 150 and/or inner skirt 140 may be fabricated to be thromboresistant. For example, a knit pattern of the material of the base skirt 150 and/or inner skirt 140 may be configured to be thromboresistant, with micropatterns or other forms of knit patterns. A knit PTFE fabric may be utilized in some examples. The material of the base skirt 150 and/or inner skirt 140 can optionally include a smooth texture to be thromboresistant and inhibit tissue growth. Combinations of such features may be utilized to result in a thromboresistant base skirt 150 and/or inner skirt 140, and/or a thromboresistant outer skirt inner surface 182.

In some examples, components of the prosthetic valve assembly 300 can optionally comprise coatings or additional structures which serve as matrices configured for the delivery of various therapeutic agents. In some examples, the frame 106 may be a drug eluting frame. In some examples, any of the inner skirt 140, base skirt 150, and/or outer skirt 170, can be a drug-eluting skirt. Suitable therapeutic agents include anti-thrombotic agents and/or anti-proliferative agents, which are effective in the prevention or inhibition of tissue growth over different surfaces of the prosthetic valve assembly, such as the frame outer surface 108, the inner surface 182 of the outer skirt 170, and/or outer surface of a first skirt, such as base skirt outer surface 160 and/or inner skirt outer surface 146. In some examples, the therapeutic agent is an angiogenesis inhibitor. Particular drugs may include but are not limited to: Batimastat, Heparin (including synthetic heparin salts), Sirolimus, Paclitaxel, and Everolimus.

The drug eluting component(s) (e.g., drug eluting frame 106, or drug eluting skirt 140, 150 and/or 170) can optionally comprise a surface coating or matrix bonding to at least a portion of a support structure. The surface coating or matrix system may have a diffusion-type, erosive-type or reservoir-based drug release mechanism. For example, a drug eluting frame 106 may include a polymeric matrix which permits elution of drugs over a period of time ranging from several hours to several months after implantation. For this purpose, polymers such as polyurethane can optionally be used in the matrix. In another example, a drug eluting skirt, such as any of skirts 140, 150, 170, may include a base layer that can be made of a fabrics or a suitable polymers, which is impregnated, entangled, coated, embedded, incorporated, encapsulated, bound, or attached to one or more of the therapeutic agents prior to implantation of prosthetic valve assembly 300 for controlled release over an extended duration.

It will be appreciated that tissue ingrowth on the outer surface 180 of the outer skirt 170 may enable stable positioning of the prosthetic valve 100 while implanted and operating in the native heart valve. It will be further appreciated that use of a thromboresistant material or layer on the inner surface 182 of the outer skirt 170 and/or outer surface of a first skirt, such as base skirt outer surface 160 and/or inner skirt outer surface 146, may provide improved separation of the outer skirt 170 from the prosthetic valve 100, and use of a thromboresistant material or layer may reduce cutting of surrounding tissue during explant.

As shown in FIG. 10, when explantation of a previously implanted prosthetic valve is required, a release device, including cutting tool 340, such as a sharp blade or scalpel illustrated in FIG. 10, an RF source, scissors, or any other suitable cutting member, or a grabber (not shown) that can be used to grab and pull on a suture, can be approximated towards one or more retaining suture(s) 330, including toward suture offset sections 334 and/or suture coupling end(s) 332 thereof, and utilized to cut the retaining suture(s) 330 or otherwise release a suture coupling end 332 thereof (such as by unraveling a knot by a grabber), after which the loose end(s) of the retaining suture(s) 330 can be pulled to separate between the outer skirt 170 and the remainder of prosthetic valve 100, in a manner that allows easier removal of the prosthetic valve 100 while the outer skirt 170 can remain attached to the surrounding heart valve annulus. In some examples, the cutting tool 340 can optionally be provided with a relatively blunt, rounded, and/or atraumatic tip 342, such that when approximated to the retaining suture(s) 330, optionally from the inner side of the frame 106, the tip will not accidentally cut other soft components of the prosthetic valve, and reduce the risk of scratching or otherwise damaging other components of the prosthetic valve.

A knot 332, or any attachment method of the retaining suture 330, can optionally be formed at any appropriate region of the frame 106, such as proximal to the first and the second skirts as shown for knot 332a, or distal to the first and the second skirts as shown for knot 332b. In some examples, when more than one retaining suture 330 is provided, each can optionally be tied or otherwise attached to a different portions of the frame 106, such as a first knot 332a and a second knot 332b tied to axial opposite regions of the frame 106 relative to the first and the second skirts. While two knots 332a and 332b are shown in combination in FIG. 10, it is to be understood that this is not meant to be limiting, and that any prosthetic valve assembly 300 can optionally include a single retaining suture 330 having a single knot 332 tied to any appropriate region of the frame, such as only proximal to the first and the second skirts or only distal to the first and the second skirts, or it can optionally include a plurality of retaining sutures 330 with their knots 332 tied to any other appropriate position of the frame 106.

In some examples, a removable skirt can optionally be provided with a prosthetic valve, without being connected to another skirt, such as to a base skirt or an inner skirt. The removable skirt can optionally be an outer skirt 170 that can be coupled, by at least one skirt removal device, such as a retaining suture 330, directly to the frame 106 or components attached to the frame.

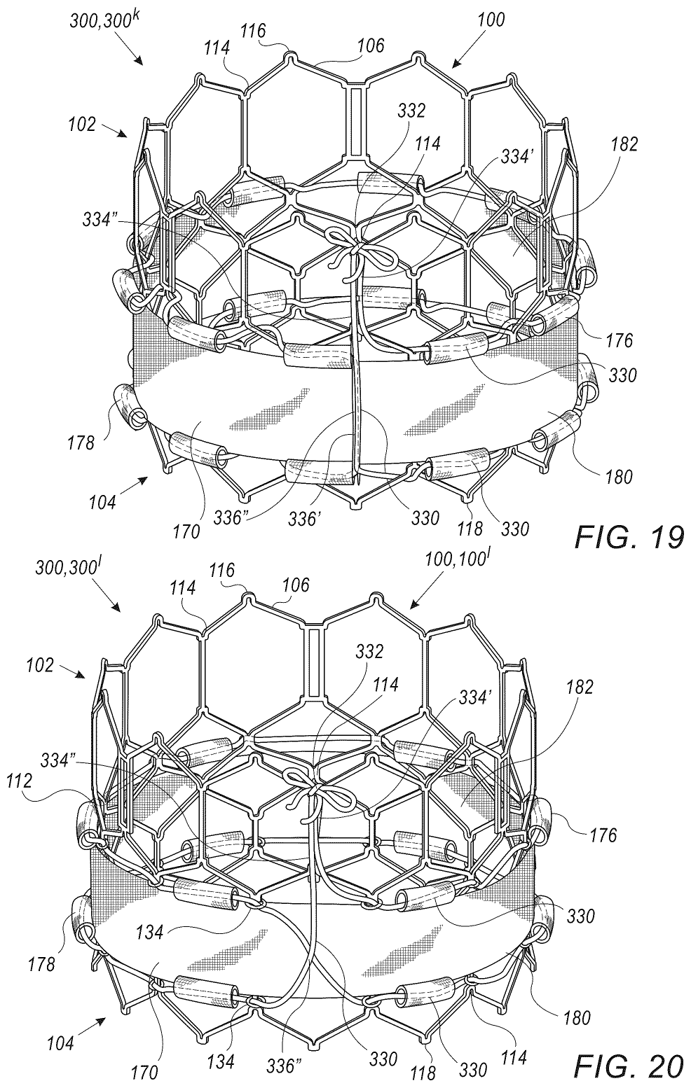

FIG. 19 is a view in perspective of an exemplary prosthetic valve assembly 300^{k}. Prosthetic valve assembly 300^{k} is an exemplary implementation of prosthetic valve assembly 300, and thus includes all of the features described for prosthetic valve assembly 300 throughout the current disclosure, except that it comprises an outer skirt 170 which is coupled, by a retaining suture 330, to the frame 106. In some examples, prosthetic valve assembly 300^{k} can be devoid of any other additional skirts, such as a base skirt or an inner skirt. In some examples, the prosthetic valve 300^{k} can optionally include at least one additional skirt, such as an inner skirt 140, wherein the outer skirt 170 is coupled by the retaining suture 330 to the frame 106 but not to the inner skirt 140. As shown, the retaining suture 330 can optionally pass through the outer skirt outflow loops 176 and inflow loops 178, and weaved around struts 112 and/or junctions 114 of the frame 106 along portions disposed between adjacent loops 176, 178 of the outer skirt 170.

FIG. 20 is a view in perspective of an exemplary prosthetic valve assembly 300^{l}. Prosthetic valve assembly 300^{l} is an exemplary implementation of prosthetic valve assembly 300, and thus includes all of the features described for prosthetic valve assembly 300 throughout the current disclosure, except that it comprises an outer skirt 170 which is coupled, by a retaining suture 330, to the frame 106 of prosthetic valve 100^{l}. Prosthetic valve 100^{l} can be similar to any exemplary prosthetic valve 100 disclosed herein, except that prosthetic valve 100^{l} can further include eyelets 134 attached to the frame 106. The eyelets 134 can optionally be attached to junctions 114 and/or struts 112 of the frame 106. In some examples, prosthetic valve assembly 300^{l} can optionally be devoid of any other additional skirts, such as a base skirt or an inner skirt. In some examples, the prosthetic valve 300^{l} can optionally include at least one additional skirt, such as an inner skirt 140, wherein the outer skirt 170 is coupled by the retaining suture 330 to the frame 106 but not to the inner skirt 140. As shown, the retaining suture 330 can optionally pass through the outer skirt outflow loops 176 and inflow loops 178, and through eyelets 134 disposed at portions of the frame that reside between adjacent loops 176, 178 of the outer skirt 170.

In some cases, it may be desirable to provide a prosthetic valve kit that includes a prosthetic valve implantable with a skirt removal device coupled thereto. In some examples, the skirt removal device of a prosthetic valve kit is a cutting member. The kit is implantable inside a patient's body, with the cutting member residing next to the prosthetic valve and can be used to facilitate explant procedures if desired, at which point the cutting member can be utilized to separate between the prosthetic valve and the surrounding anatomy and/or between the outer skirt of a prosthetic valve and the remainder of the valve.

Described herein are examples of prosthetic valve kits 350 and components thereof (shown, for example, in FIGS. 11A-15). A prosthetic valve kit 350 comprises a prosthetic valve 100 and a cutting member 360. A cutting member 360 includes, as shown for example in FIGS. 11A-15, an elongated segment 362 extending from a proximal end portion 364 to a distal end portion 366. The cutting member 360 comprises at least one terminal 370, which can optionally be provided, in various examples, at its proximal end portion 364 and/or its distal end portion 366. The cutting member 360 can optionally be coupled to the prosthetic valve 100 such that the elongated segment 362 is disposed radially outward of the frame 106, and optionally such that the elongated segment 362 extends parallel to the central longitudinal axis Ca.

FIG. 11A shows an exemplary cutting member 360^{a}. Cutting member 360^{a} is an exemplary implementation of cutting member 360, and thus includes all of the features described for cutting member 360 throughout the current disclosure, except that cutting member 360^{a} further comprises a circumferential segment 368^{a} extending from its distal end portion 366^{a}. In some examples, circumferential segment 368^{a} defines a plane that is generally perpendicular to an axis defined by elongated segment 362, and/or to longitudinal axis Ca of the prosthetic valve 100 when coupled thereto. In some examples, circumferential segment 368^{a} is shaped as a full ellipse or circle. In some examples, circumferential segment 368 is a closed shape.

As mentioned above, cutting member 360 further includes at least one terminal 370. For example, and as illustrated in FIG. 11A, cutting member 360^{a} includes a terminal 370 provided at its proximal end portion 364. Terminal 370 can be generally rectangular in shape as illustrated, though it is to be understood that any other shape is contemplated.

In some examples, cutting member 360 comprises an electrically conductive material such that electrical current can flow from terminal 370 to another suitable portion of the cutting member 360. For example, cutting member 360^{a} can optionally comprise an electrically conductive material such that electrical current can flow from terminal 370 to circumferential segment 368. In some examples, at least a portion of elongated segment 362 is covered with an electrically insulative surface.

In some examples, electric current can optionally be provided to a cutting member 360 of a previously implanted prosthetic valve kit 350 by a transcatheter delivery apparatus or assembly, including, but not limited to, by a delivery apparatus 202 of the type described above with respect to new prosthetic valve implantation. In some examples, electric current can optionally be provided to a cutting member 360 of a previously implanted prosthetic valve kit 350 by attachment of a power transfer device 380 (shown, for example, in FIG. 12B) to terminal 370.

FIG. 2 illustrates one example of a delivery apparatus 202 that can optionally include an electric current source 205 configured to generate and output a predetermined electric current. In some examples, as schematically illustrated in FIG. 2, electric current source 205 is secured within handle 204. In some examples, electric current source 205 can optionally be external to handle 204. In some examples, electric current source 205 can optionally be external to the patient's body, and is not necessarily part of a delivery apparatus but rather part of a stand-alone system configured to provide electric current via a power transfer device 380 that can optionally be attachable to terminal 370.

In some examples, electric current source 205 generates an electric current. In some examples, the generated electric current is an alternating current. In some examples, the frequency of the alternating current is a radio-frequency (RF), i.e. from about 20 kHz to 300 GHz. In some examples, at least a portion of cutting member 360, such as circumferential segment 368 of cutting member 360^{a}, is configured to output RF energy.

In some examples, electric current source 205 is configured, responsive to a user input, to alternately provide the electric current and not provide the electric current. For example, electric current source 205 can optionally comprise a user input device (not shown), such as a switch that can turn the electric current source on and off. Thus, as will be described below, the electric current can optionally be provided only when it is desired to use cutting member 360, for example to cut away outer skirt 170.

FIG. 11B shows a cross-sectional view of elongated element 362 of cutting member 360^{a} and FIG. 11C shows a cross-sectional view of circumferential segment 368 of cutting member 360^{a}. In some examples, as illustrated in FIG. 11B, cutting member 360^{a} comprises 3 layers: electrically conductive inner and outer layers 361; and a non-conductive layer 363 positioned between inner and outer layers 361. Although they are positioned as separate layers, inner layer 361 and outer layer 361 are an extension of each other and provide a continuous conductive path.

FIGS. 12A-12B shows steps in the utilization of a cutting member 360^{a} of an exemplary prosthetic valve kit 350^{g}. FIG. 12A shows a sectional side view of prosthetic valve kit 350^{g} prior to providing electric current to cutting member 360^{a}. This view can be representative, for example, of the configuration of the kit 350^{g} as implanted in a patient's body, prior to initiating an explantation procedure. Prosthetic valve kit 350^{g} is an exemplary implementation of prosthetic valve kit 350, and thus includes all of the features described for prosthetic valve kit 350, except that the prosthetic valve kit 350^{g} includes a cutting member 360^{a} disposed, at least along a portion thereof, between the frame 106 and the outer skirt 170 of prosthetic valve 100^{g}.

As shown in FIG. 12A, elongated segment 362^{a} of cutting member 360^{a} can optionally extend, at least along a portion thereof, between the frame 106 and the outer skirt 170, such as, in some examples, between the frame outer surface 108 and the outer skirt inner surface 182. Prosthetic valve 100^{g} is an exemplary implementation of prosthetic valve 100, and thus includes all of the features described for prosthetic valve, except that the outer skirt 170 of prosthetic valve 100^{g} can optionally be coupled to the frame 106 and/or to inner skirt 140 by outer skirt connection suture 192, while the inner skirt 140 can optionally be coupled to the frame 106 by inner skirt connection suture 190, which can optionally be separately formed from outer skirt connection sutures 192.

As illustrated in FIG. 12A, the elongated segment 362^{a} can optionally be sandwiched between the frame 106 and outer skirt 170, optionally between outer skirt connection sutures 192. While two suture lines of outer skirt connection sutures 192 are illustrated in FIG. 12A, such as outer skirt distal connection sutures 192a positioned closer to the outer skirt inflow end 174, and outer skirt proximal connection sutures 192b positioned closer to the outer skirt outflow end 172, it is to be understood that this is shown by way of illustration and not limitation, and that any other arrangement of outer skirt connection sutures 192 is contemplated.

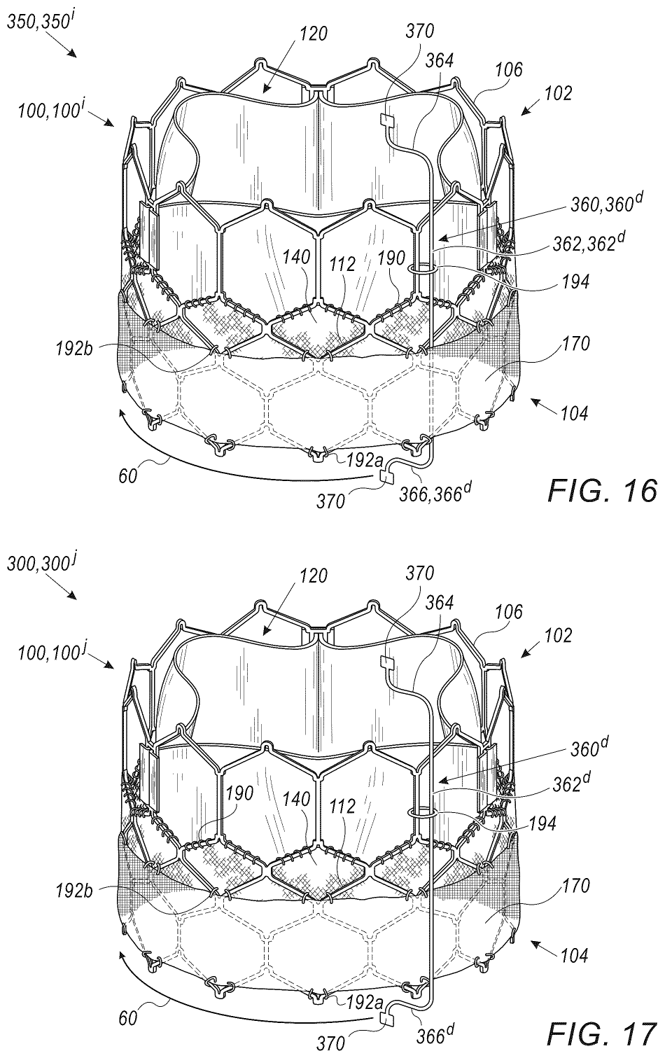

Optionally, additional couplers can be utilized to couple the cutting member 360 to the prosthetic valve 100. For example, the kit 350 can optionally further include one or more cutting member couplers 194 (not shown in FIGS. 12A-B, but an example of which is illustrated in FIGS. 16-17), that can be used to couple the cutting member 360 to the frame 106 at least temporarily. A cutting member coupler 194 can optionally be provided, in some examples, in the form of a loop, such as a suture loop (as illustrated in FIGS. 16-17). In some examples, cutting member coupler 194 can optionally be cut prior to and/or during utilization of cutting member 360. In some examples, electric current flowing through cutting member 360 can optionally be utilized also to cut through cutting member coupler 194. In some examples, cutting member coupler 194 is separately cut, such as by scissor, blade, or any other tool (e.g., cutting tool 340), to allow undisturbed movement of the cutting member 360 during utilization thereof.

In some examples, the inner diameter of the circumferential segment 368 is substantially equal to the outer diameter of the frame 106 in the expanded configuration, such that the circumferential segment 368 can circumscribe the frame 106. In the example illustrated in FIG. 12A, the cutting member 360a is coupled to the prosthetic valve 100 such that the circumferential segment 368 is proximate to the inflow end 104 of the frame 106, optionally distal to the outer skirt inflow end 174 and/or distal to outer skirt connection sutures 192, such as outer skirt distal connection sutures 192a.

In some examples, a power transfer device 380 is provided that can be attached to, and detached from, terminal 370. In some examples, power transfer device 380 comprises an alligator clip that can be attached to terminal 370, though any other suitable attachment interface is contemplated. In some examples, power transfer device 380 is permanently connected to electric current source 205. Alternatively, power transfer device 380 can optionally be attached to, and detached from, electric current source 205.

In the state illustrated in FIG. 12B, power transfer device 380 has been connected to terminal 370 to provide electric current, generated by electric current source 205, to terminal 370. In some examples, an end portion of the cutting member 360, such as an end portion that includes terminal 370, includes a bent portion, such as being bent or extended radially inwards (i.e., towards central longitudinal axis Ca), to offset the terminal farther away from the surrounding anatomy (e.g., blood vessel wall), thereby providing easier access for connecting a power transfer device 380 to the terminal 370. Moreover, such bend or inwardly-oriented extension can facilitate easier grasping (e.g., manual grasping) of the cutting member 360, for example to facilitate axial movement thereof as will be described below. In the illustrated example, the proximal end portion 364 of cutting member 360^{a} is shown to extend radially inwards, relative to the elongated segment 362^{a}.

As mentioned, cutting member 360 comprises an electrically conductive material, such that the electric current flows from terminal 370, along elongated segment 362^{a}, to circumferential segment 368. Elongated segment 362^{a}, as further mentioned above, can optionally be covered with an electrically insulative surface, such that the conductive core of elongated segment 362^{a} is insulated from frame 106 and outer skirt 170, as well as any other component of prosthetic valve 100 or anatomical features contacted by elongated segment 362^{a}, during current transfer toward circumferential segment 368.

In contrast, the circumferential segment 368 is formed of an electrically conductive material which is not covered by an insulated surface, exposing it to the external environment as current flows therethrough. As further shown in FIG. 12B, the cutting member 360^{a} can be then axially pulled, such as in a proximal direction 50, causing the circumferential segment 368 to axially slide in the same direction between the frame 106 and the outer skirt 170, forcing it to contact, during this axial movement, the outer skirt connection sutures 192. The electric current flowing through circumferential segment 368 is configured to generate heat, which is sufficient to cut through the outer skirt connection sutures 192, such that pulling the cutting member 360^{a} and its circumferential segment 368 serves to cut through the sutures 192 connecting the outer skirt 170 to the frame 106, thus separating between the outer skirt 170 and the remainder of prosthetic valve 100.

As further shown in the illustrated example, the inner skirt 140 can remain sutured to the frame 106 since it is attached to the frame 106 by a separate set of inner skirt connecting sutures 190, which can optionally remain intact. In some examples, inner skirt connecting sutures 190 are made from a different material than that of the outer skirt connection sutures 192, and/or are covered by a protective cover and/or coated by a protective coating, so as to protect sutures 190 from being cut by circumferential segment 368 as it is axially passed to cut through sutures 192.

Once all outer skirt connection sutures 192 are cut, the outer skirt 170 is separated from frame 106. The power transfer device 380 can be optionally disconnected from terminal 370, and the prosthetic valve 100 and cutting member 360 can be removed from the patient's body, while the outer skirt 170 can optionally remain attached to the surrounding anatomy, for example due to tissue overgrowth between the skirt 170 and the surrounding annulus and/or blood vessel walls.

While the circumferential segment 368 is illustrated in FIG. 12A, in the assembled configuration of kit 350^{g}, to be positioned distal to the outer skirt inflow end 174 and/or distal to outer skirt distal connection sutures 192a, it is to be understood that any other axial position of the circumferential segment 368 is contemplated. For example, circumferential segment 368 can be axially positioned, when assembled to prosthetic valve 100, between the outer skirt inflow end 174 and the outer skirt outflow end 172, and optionally between outer skirt distal connection sutures 192a and outer skirt proximal connection sutures 192b. In such examples, the cutting member 360^{a} can be similarly utilized, except that its axial movement can include both pushing in a distal direction to cut through between outer skirt distal connection sutures 192a, and pulling in a proximal direction to cut through outer skirt proximal connection sutures 192b.

While described and illustrated in combination with prosthetic valve 100, it is to be understood that any exemplary cutting member 360 described herein can be similarly utilized in combination with a prosthetic valve assembly 300. That is to say, any exemplary prosthetic valve kit 350 described herein with respect to FIGS. 12A-15, can optionally include a cutting member 360 coupled to a prosthetic valve assembly 300, and utilized to separate a prosthetic valve 100 of any exemplary prosthetic valve assembly 300 described above, in a similar manner to that described herein for use with prosthetic valve 100, mutatis mutandis.

For example, a prosthetic valve kit 350^{g} can be implemented in a similar manner to that described above with respect to FIGS. 12A-12B, except that when used with a prosthetic valve assembly 300, the cutting member 360^{a} can be disposed between the first skirt and the second skirt, and utilized in a similar manner, to cut through appropriate portions of the one or more retaining sutures 330, such as to cut suture coupling ends 332 and/or to cut through suture offset sections 334, as the cutting member 360^{a} and its circumferential segment 368 are axially passed between the first skirt and the second skirt.

In some examples, as mentioned, electric current source 205 can initially be switched off, and can optionally be activated prior to cutting member 360^{a} being axially pulled.

FIG. 13 shows a sectional side view of prosthetic valve kit 350^{h}. Prosthetic valve kit 350^{h} is an exemplary implementation of prosthetic valve kit 350, and thus includes all of the features described for prosthetic valve kit 350, and in particular, can include a cutting member 360^{a} coupled to prosthetic valve 100^{h} in a manner similar to that described above with respect to prosthetic valve kit 350^{g}, except that the cutting member 360^{a} is disposed radially outward to outer skirt 170. Prosthetic valve 100^{h} can be similar to prosthetic valve 100^{g}, except that the outer skirt 170 can be tightly disposed around the frame 106, and not necessarily define a gap between the skirt 170 and the frame 106 through which the cutting member 360 can extend.

In some examples, the inner diameter of the circumferential segment 368 is substantially equal to the outer diameter of the outer skirt 170 in the expanded configuration of the frame 106, such that the circumferential segment 368 can circumscribe the outer skirt 170.

When prosthetic valve kit 350^{h} is implanted in a patient's body, the cutting member 360^{a} can optionally be disposed, at least along a portion thereof, between the outer skirt 170 and the surrounding anatomy (e.g., native annulus and/or blood vessel wall). The cutting member 360^{a} of prosthetic valve kit 350^{h} can be utilized in a similar manner to that described above, for example with respect to FIG. 12B, expect that the cutting member 360^{a} of prosthetic valve kit 350^{h} serves to cut through the tissue ingrowth around prosthetic valve 100^{h}, so as to cut between the surrounding tissue and the outer skirt 170.

The cutting member 360^{a} of prosthetic valve kit 350^{h} can optionally serve as a diathermic cutting tool. In some examples, the frequency of the electric current generated by electric current source 205 is from about 400 kHz to 10 MHz. In some examples, the amplitude of the current generated by electric current source 205 is up to 500 milliamperes (mA). In some examples, the absolute value of the voltage of the current generated by electric current source 205 is at least 200 Volts. The electric current, flowing through circumferential segment 368, which is not insulated but rather exposed to the surrounding environment, generates heat when it passes through the tissue, which serves in turn to cut through the surrounding tissue. In some examples, the electric current flowing through circumferential segment 368 generates heat of up to 1000 degrees C. When the cutting member 360^{a} is axially pulled in a manner similar to that described above, the circumferential segment 368 cuts through the tissue that surrounds the outer skirt 170, thereby separating the prosthetic valve 100 and its outer skirt 170 from the surrounding tissue, after which the power transfer device 380 can optionally be optionally disconnected from terminal 370, and the prosthetic valve 100 and cutting member 360 can optionally be removed from the patient's body.

Compared with prosthetic valve kit 350^{g} described above with respect to FIGS. 12A-12B, enabling removal of prosthetic valve 100^{g} while outer skirt 170, cut away therefrom, remains adhered to the patient's tissue, the prosthetic valve kit 350^{h} enables removal of prosthetic valve 100^{h} together with the outer skirt 170, leaving a greater open space in which a guest valve can optionally be then implanted, absent of the additional layer of a skirt 170 that would have otherwise narrowed to some degree the available space for subsequent implantation.

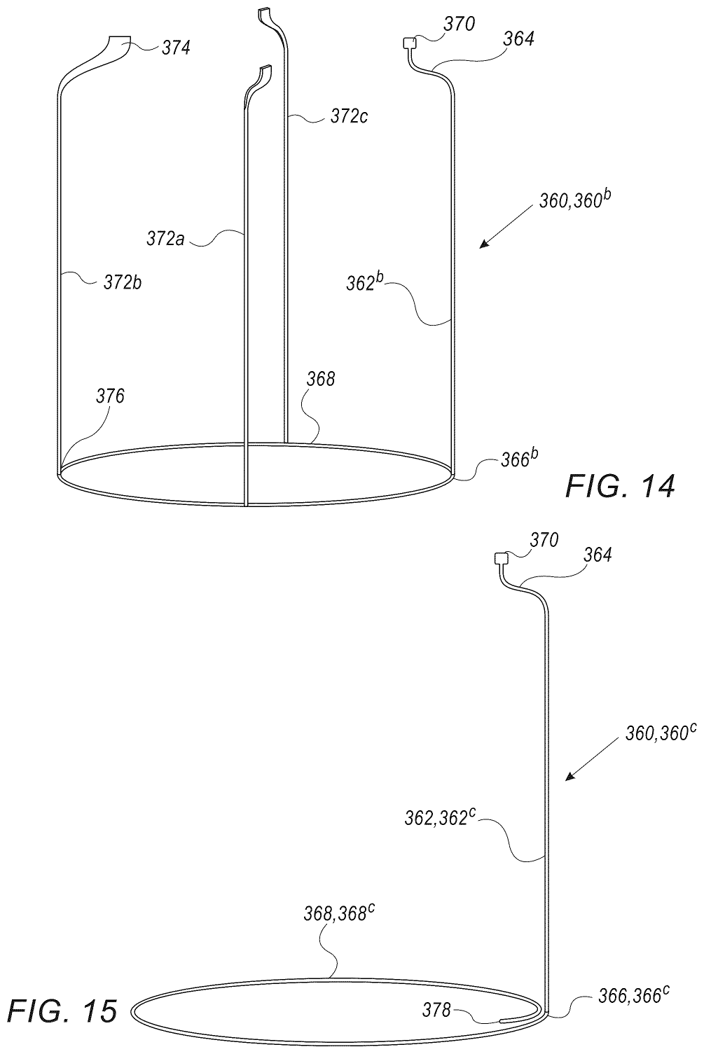

FIG. 14 illustrates an exemplary cutting member 360^{b}. Cutting member 360^{b} is an exemplary implementation of cutting member 360, and thus includes all of the features described for cutting member 360 throughout the current disclosure, except that cutting member 360^{b} further comprises one or more additional elongated segments 372. Cutting member 360^{b} is illustrated herein as comprising additional elongated segments 372a, 372b and 372c, however this is not meant to be limiting in any way, and cutting member 360^{b} can optionally comprise any number of additional elongated segments 372, including a single additional elongated segment 372.

Each additional elongated segment 372 extends from a proximal end 374 to a distal end 376. In some examples, each elongated segment 372 extends proximally from a respective portion of circumferential segment 368, at the distal end 376. In some examples, proximal ends 374 are shaped such that they each have a larger surface area than that of terminal 370 of elongated segment 362. In some examples (not shown), each proximal end 374 has a respective terminal 370 extending therefrom. In some examples, additional elongated segments 372 can optionally be utilized to provide additional connection points for a mechanism that pulls cutting member 360^{b} proximally. In some examples, this can prevent circumferential segment 368 from becoming angularly locked due to proximal pulling from only a single point.

FIG. 15 illustrates an exemplary cutting member 360^{c}. Cutting member 360^{c} is an exemplary implementation of cutting member 360, and thus includes all of the features described for cutting member 360 throughout the current disclosure, except that cutting member 360^{c} comprises a circumferential segment 368^{c}. In some examples, circumferential segment 368^{c} is an open shape. In some examples, circumferential segment 368^{c} is shaped as at least a portion of an ellipse. In some examples, circumferential segment 368c has an end 378 extending past distal portion 366 of elongated segment 362. In some examples, this can prevent circumferential segment 368 from becoming angularly locked due to proximal pulling of circumferential segment 368. In some examples (not shown), end 378 of circumferential segment 368^{c} does not extend that far. For example, circumferential segment 368 can optionally extend around any fraction of the circumference of the frame 106.

FIG. 16 shows a view in perspective of prosthetic valve kit 350ⁱ. Prosthetic valve kit 350ⁱ is an exemplary implementation of prosthetic valve kit 350, and thus includes all of the features described for prosthetic valve kit 350, and in particular, can include a cutting member 360^{a} coupled to prosthetic valve 100^{h} in a manner similar to that described above with respect to prosthetic valve kit 350^{g}, except that the prosthetic valve kit 350ⁱ includes a cutting member 360^{d} disposed, at least along a portion thereof, between the frame 106 and the outer skirt 170 of prosthetic valve 100ⁱ (which can be identical to prosthetic valve 100^{g}). Cutting member 360^{d} is an exemplary implementation of cutting member 360, and thus includes all of the features described for cutting member 360 throughout the current disclosure, except that at least a portion of the elongated segment 362^{d} of cutting member 360^{d} is exposed (i.e., not insulated). The cutting member 360^{d} can be devoid of a circumferential loop. In some examples, a terminal 370 can optionally be provided at the distal end portion 366^{d}.

In some examples, two terminals 370 can optionally be provided at both the proximal end portion 364 and distal end portion 366^{d}, as illustrated. This can allow greater flexibility in choosing the most suitable terminal 370 to connect with, according to patient specific anatomy, for example. Nevertheless, while terminals 370 are shown at both end portions of the exemplary cutting member 360^{d} illustrated in FIGS. 16-17, it is to be understood that this is merely optional, and that in other implementations, a terminal 370 can optionally be provided only at one of the end portions. In some examples, only the proximal end portion 364 comprises a terminal 370. In some examples, only the distal end portion 366^{d} comprises a terminal.

In some examples, both of the proximal end portion 364 and distal end portion 366 are inwardly bent or include an inwardly-oriented extension, as illustrated in FIGS. 16 and 17. This can be of benefit when both end portions include terminals 370, so as to offset the terminal 370 away from the native anatomy, as mentioned above. Moreover, even when a terminal 370 is provided on only one of the end portions, both end portions can optionally be still bent or extended radially inwards, so as to facilitate easier grasping (e.g., manual grasping) of both ends of the cutting member 360^{d}, to facilitate circumferentially-oriented movement of the cutting member 360^{d} as will be described below. Nevertheless, while both end portions of the exemplary cutting member 360^{d} illustrated in FIGS. 16-17 are shown to be bent inwards, it is to be understood that this is merely optional, and that in some examples, none or only one of the end portions is bent or extended radially inwards. In some examples, only the proximal end portion 364 is bent or comprises a radial extension. In some examples, only the distal end portion 366^{b} is bent or comprises a radial extension.

As illustrated in, for example, FIGS. 16 and 17, the overall length of the elongated segment 362^{d} can optionally be greater than the height of the outer skirt 170, defined between the outer skirt inflow end 174 and outer skirt outflow end 172. The elongated segment 362^{d} is exposed (i.e., not insulated) at least along the portion disposed between the outer skirt inflow end 174 and outer skirt outflow end 172. In some examples, the elongated segment 362^{d} is exposed along its entire length, between the proximal end portion 364 and the distal end portion 366^{d}. In some examples, at least a portion of the elongated segment 362^{d} is covered with an electrically insulative surface, such as a portion of the elongated segment 362^{d} extending proximally from the outer skirt outflow end 172 or distally from the outer skirt inflow end 174.

In use, a power transfer device 380 can optionally be connected to terminal 370 (for example, to one of two terminals 370 if two are provided), and the electric current source 205 can be powered to generate electric current passing through the elongated segment 362^{d}. The elongated segment can optionally be then rotated in a circumferential direction 60 (as indicated in FIG. 16) around the frame 106, such that the exposed conductive portion of the elongated segment 362^{d} is forced to pass and cut through the outer skirt connection sutures 192 during such circumferential movement thereof. This separates the outer skirt 170 from the frame 106, allowing retrieval of the prosthetic valve 100 in a similar manner to that described above.

FIG. 17 shows a view in perspective of prosthetic valve kit 350^{j}. Prosthetic valve kit 350^{j} is an exemplary implementation of prosthetic valve kit 350, and thus includes all of the features described for prosthetic valve kit 350, and in particular, can include a cutting member 360^{d} coupled to prosthetic valve 100^{j} (which can be identical to prosthetic valve 100^{h}) in a manner similar to that described above with respect to prosthetic valve kit 350ⁱ, except that the cutting member 360^{d} is disposed radially outward to outer skirt 170.

The cutting member 360^{d} of prosthetic valve kit 350^{j} can optionally serve as a diathermic cutting tool, in a similar manner to that described above with respect to prosthetic valve kit 350^{h}. When the cutting member 360^{d} is circumferentially rotated around the outer skirt 170 in a manner similar to that described above, the elongated segment 362^{d} cuts through the tissue that surrounds the outer skirt 170, thereby separating the prosthetic valve 100 and its outer skirt 170 from the surrounding tissue, after which the power transfer device 380 can be optionally disconnected, and the prosthetic valve 100 and cutting member 360 can be removed from the patient's body.

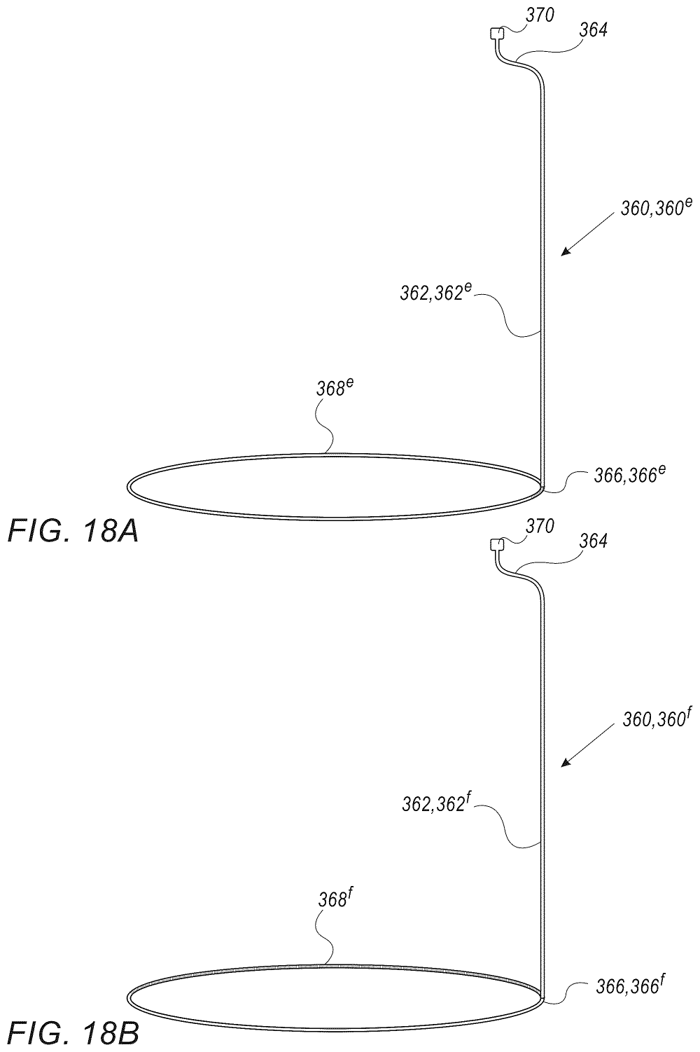

FIG. 18A illustrates an exemplary cutting member 360^{e}. Cutting member 360^{e} is an exemplary implementation of cutting member 360, and thus includes all of the features described for cutting member 360 throughout the current disclosure, except that circumferential segment 368^{e} comprises an electrically isolated portion to avoid presenting a short circuit, as known to those skilled in the art.

FIG. 18B illustrates an exemplary cutting member 360^{f}. Cutting member 360^{f} is an exemplary implementation of cutting member 360, and thus includes all of the features described for cutting member 360 throughout the current disclosure, except that at least a portion of the inner face of circumferential segment 368^{f} is electrically isolated such that an electric current flowing through circumferential segment 368^{f} does not flow through frame 106, as known to those skilled in the art.

### SOME EXAMPLES OF THE DISCLOSED IMPLEMENTATIONS

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve assembly comprising: a prosthetic valve comprising: an annular frame movable between a radially compressed and a radially expanded configuration; and a removable skirt disposed radially outward to the frame, the removable skirt comprising one or more inflow loops extending from an inflow end of the removable skirt, and one or more outflow loops extending from an outflow end of the removable skirt; and at least one retaining suture coupled to the frame at a suture coupling end, and extending through the one or more inflow loops of the removable skirt, and through the one or more outflow loops of the removable skirt; wherein the one or more inflow loops of the removable skirt and the one or more outflow loops of the removable skirt are arranged around the circumference of the frame; and wherein the removable skirt is configured to be released from the frame upon withdrawal of the at least one retaining suture from the one or more inflow loops of the removable skirt and from the one or more outflow loops of the removable skirt.

Example 2. The prosthetic valve assembly of any example herein, particularly example 1, wherein the removable skirt is an outer skirt.

Example 3. The prosthetic valve assembly of any example herein, particularly example 1 or 2, wherein the one or more inflow loops of the removable skirt comprises a plurality of inflow loops of the removable skirt, arranged around the circumference of the frame, and wherein the one or more outflow loops of the removable skirt comprises a plurality of outflow loops of the removable skirt, arranged around the circumference of the frame.

Example 4. The prosthetic valve assembly of any example herein, particularly example 3, wherein the at least one retaining suture is weaved around struts of the frame between adjacent inflow loops of the plurality of inflow loops of the removable skirt, and around struts of the frame between adjacent outflow loops of the plurality of outflow loops of the removable skirt.

Example 5. The prosthetic valve assembly of any example herein, particularly example 3, wherein the prosthetic valve further comprises a plurality of eyelets attached to the frame.

Example 6. The prosthetic valve assembly of any example herein, particularly example 5, wherein the at least one retaining suture is passed through at least some of the plurality of eyelets.

Example 7. The prosthetic valve assembly of any example herein, particularly example 1, wherein the prosthetic valve further comprises a first skirt disposed around the frame and coupled to the frame, the first skirt comprising one or more inflow loops extending from an inflow end of the first skirt, and one or more outflow loops extending from an outflow end of the first skirt; wherein the removable skirt is a second skirt; wherein the at least one retaining suture extends through the one or more inflow loops of the first and the second skirts, and through the one or more outflow loops of the first and the second skirts; wherein the one or more inflow loops of the first skirt and of the second skirt are arranged in an alternating manner around the circumference of the frame; wherein the one or more outflow loops of the first skirt and of the second skirt are arranged in an alternating manner around the circumference of the frame; and wherein the second skirt is configured to be released from the first skirt upon withdrawal of the at least one retaining suture from the one or more inflow loops of the first and the second skirts and from the one or more outflow loops of the first and the second skirts.

Example 8. The prosthetic valve assembly of any example herein, particularly example 7, wherein the one or more inflow loops of the first skirt comprises a plurality of inflow loops of the first skirt.

Example 9. The prosthetic valve assembly of any example herein, particularly example 7 or 8, wherein the one or more outflow loops of the first skirt comprises a plurality of outflow loops of the first skirt.

Example 10. The prosthetic valve assembly of any example herein, particularly any one of examples 7 to 9, wherein the one or more inflow loops of the second skirt comprises a plurality of inflow loops of the second skirt.

Example 11. The prosthetic valve assembly of any example herein, particularly any one of examples 7 to 10, wherein the one or more outflow loops of the second skirt comprises a plurality of outflow loops of the second skirt.

Example 12. The prosthetic valve assembly of any example herein, particularly any one of examples 7 to 11, wherein the second skirt is an outer skirt of the prosthetic valve assembly, wherein the inflow end of the second skirt is an outer skirt inflow end, wherein the outflow end of the second skirt is an outer skirt outflow end, wherein the one or more inflow loops of the second skirt is one or more outer skirt inflow loops, and wherein the one or more outflow loops of the second skirt is one or more outer skirt outflow loops.

Example 13. The prosthetic valve assembly of any example herein, particularly example 12, wherein the first skirt is a base skirt disposed around an outer surface of the frame, wherein the inflow end of the first skirt is a base skirt inflow end, wherein the outflow end of the first skirt is a base skirt outflow end, wherein the one or more inflow loops of the first skirt is one or more base skirt inflow loops, and wherein the one or more outflow loops of the first skirt is one or more base skirt outflow loops.

Example 14. The prosthetic valve assembly of any example herein, particularly example 13, wherein the base skirt is sutured to the frame.

Example 15. The prosthetic valve assembly of any example herein, particularly example 13 or 14, wherein the prosthetic valve further comprises an inner skirt disposed around an inner surface of the frame.

Example 16. The prosthetic valve assembly of any example herein, particularly example 15, wherein the inner skirt is sutured to the frame.

Example 17. The prosthetic valve assembly of any example herein, particularly example 15 or 16, wherein the base skirt is sutured to the inner skirt.

Example 18. The prosthetic valve assembly of any example herein, particularly example 12, wherein the first skirt is an inner skirt disposed around an inner surface of the frame, wherein the inflow end of the first skirt is an inner skirt inflow end, wherein the outflow end of the first skirt is an inner skirt outflow end, wherein the one or more inflow loops of the first skirt is one or more inner skirt inflow loops, and wherein the one or more outflow loops of the first skirt is one or more inner skirt outflow loops.

Example 19. The prosthetic valve assembly of any example herein, particularly example 18, wherein the inner skirt is sutured to the frame.

Example 20. The prosthetic valve assembly of any example herein, particularly any one of examples 7 to 19, wherein the inflow loops of the first skirt are circumferentially aligned with the inflow loops of the second skirt.

Example 21. The prosthetic valve assembly of any example herein, particularly any one of examples 7 to 20, wherein the outflow loops of the first skirt are circumferentially aligned with the outflow loops of the second skirt.

Example 22. The prosthetic valve assembly of any example herein, particularly any one of examples 7 to 21, wherein a height of the first skirt is equal to a height of the second skirt.

Example 23. The prosthetic valve assembly of any example herein, particularly any one of examples 7 to 22, wherein the inflow end of the first skirt is axially aligned with the inflow end of the second skirt.

Example 24. The prosthetic valve assembly of any example herein, particularly any one of examples 7 to 23, wherein the outflow end of the first skirt is axially aligned with the outflow end of the second skirt.

Example 25. The prosthetic valve assembly of any example herein, particularly any one of examples 7 to 24, wherein the suture coupling end comprises a knot at which the at least one retaining suture is tied to the frame.

Example 26. The prosthetic valve assembly of any example herein, particularly any one of examples 7 to 25, wherein the suture coupling end is axially offset from the second skirt.

Example 27. The prosthetic valve assembly of any example herein, particularly any one of examples 7 to 26, wherein the suture coupling end is positioned between the outflow end of the second skirt and an outflow end of the frame.

Example 28. The prosthetic valve assembly of any example herein, particularly any one of examples 7 to 24, wherein the suture coupling end is axially offset from the first skirt.

Example 29. The prosthetic valve assembly of any example herein, particularly example 28, wherein the suture coupling end is positioned between the outflow end of the first skirt and an outflow end of the frame.

Example 30. The prosthetic valve assembly of any example herein, particularly any one of examples 7 to 29, wherein the at least one retaining suture comprises a single retaining suture.

Example 31. The prosthetic valve assembly of any example herein, particularly example 30, wherein the retaining suture defines a distally extending suture offset section between the suture coupling end and the outflow loops of the first and the second skirts, defines a distally extending suture crossing section between the outflow loops of the first and the second skirts and the inflow loops of the first and the second skirts, and defines a proximally extending suture crossing section extending from the inflow loops of the first and the second skirts, and a proximally extending suture offset section continuous with the proximally extending suture crossing section and terminating at the suture coupling end.

Example 32. The prosthetic valve assembly of any example herein, particularly example 31, wherein the distally extending suture crossing section and the proximally extending suture crossing section are disposed over a surface of the second skirt.

Example 33. The prosthetic valve assembly of any example herein, particularly example 31 or 32, wherein the distally extending suture crossing section and the proximally extending suture crossing section are parallel to each other.

Example 34. The prosthetic valve assembly of any example herein, particularly any one of examples 31 to 33, wherein the distally extending suture offset section and the proximally extending suture offset section are parallel to each other.

Example 35. The prosthetic valve assembly of any example herein, particularly any one of examples 31 to 34, wherein the distally extending suture crossing section and the proximally extending suture crossing section extend at an angular orientation relative to a longitudinal axis of the prosthetic valve.

Example 36. The prosthetic valve assembly of any example herein, particularly example 35, wherein the angular orientation is between 30 and 60 degrees.

Example 37. The prosthetic valve assembly of any example herein, particularly example 35, wherein the angular orientation is between 40 and 50 degrees.

Example 38. The prosthetic valve assembly of any example herein, particularly example 35, wherein the angular orientation is about 45 degrees.

Example 39. The prosthetic valve assembly of any example herein, particularly any one of examples 35 to 38, wherein the distally extending suture offset section and the proximally extending suture offset section extend at the same angular orientation as that of the distally extending suture crossing section and the proximally extending suture crossing section.

Example 40. The prosthetic valve assembly of any example herein, particularly any one of examples 7 to 29, wherein the at least one retaining suture comprises a first retaining suture and a second retaining suture.

Example 41. The prosthetic valve assembly of any example herein, particularly example 40, wherein the first retaining suture extends through the inflow loops of the first and the second skirts, but not through the outflow loops of the first and the second skirts, and wherein the second retaining suture extends through the outflow loops of the first and the second skirts, but not through the inflow loops of the first and the second skirts.

Example 42. The prosthetic valve assembly of any example herein, particularly example 41, wherein the first retaining suture defines two suture offset sections between its suture coupling end and the outflow loops of the first and the second skirts, and wherein the second retaining suture defines two suture offset sections extending from its suture coupling end, and two suture crossing sections continuous with the respective suture offset sections of the second retaining suture and extending therefrom towards the inflow loops of the first and the second skirts.

Example 43. The prosthetic valve assembly of any example herein, particularly example 42, wherein the suture crossing sections of the first and second retaining sutures are disposed over a surface of the second skirt.

Example 44. The prosthetic valve assembly of any example herein, particularly example 42 or 43, wherein the two suture crossing sections of the second retaining suture are parallel to each other.

Example 45. The prosthetic valve assembly of any example herein, particularly any one of examples 42 to 44, wherein the two suture offset sections of the first retaining suture are parallel to each other.

Example 46. The prosthetic valve assembly of any example herein, particularly any one of examples 42 to 45, wherein the two suture offset sections of the second retaining suture are parallel to each other.

Example 47. The prosthetic valve assembly of any example herein, particularly any one of examples 42 to 46, wherein the two suture offset sections of the second retaining suture and the two suture offset sections of the second retaining suture are parallel to each other.

Example 48. The prosthetic valve assembly of any example herein, particularly any one of examples 42 to 47, wherein the two suture crossing sections of the second retaining suture extend at an angular orientation relative to a longitudinal axis of the prosthetic valve.

Example 49. The prosthetic valve assembly of any example herein, particularly example 48, wherein the angular orientation is between 30 and 60 degrees.

Example 50. The prosthetic valve assembly of any example herein, particularly example 48, wherein the angular orientation is between 40 and 50 degrees.

Example 51. The prosthetic valve assembly of any example herein, particularly example 48, wherein the angular orientation is about 45 degrees.

Example 52. The prosthetic valve assembly of any example herein, particularly any one of examples 48 to 51, wherein the two suture offset sections of the second retaining suture extend at the same angular orientation as that of the two suture crossing sections of the second retaining suture.

Example 53. The prosthetic valve assembly of any example herein, particularly any one of examples 7 to 52, wherein an outer surface of the second skirt is configured to encourage tissue ingrowth.

Example 54. The prosthetic valve assembly of any example herein, particularly example 53, wherein the outer surface of the second skirt comprises yarns extending radially outward.

Example 55. The prosthetic valve assembly of any example herein, particularly example 53, wherein the outer surface of the second skirt comprises a plush layer of a fabric.

Example 56. The prosthetic valve assembly of any example herein, particularly any one of examples 7 to 55, wherein an inner surface of the second skirt is thromboresistant.

Example 57. The prosthetic valve assembly of any example herein, particularly example 56, wherein the inner surface of the second skirt comprises one or more of: PTFE, ePTFE, and/or UHMWPE.

Example 58. The prosthetic valve assembly of any example herein, particularly example 56, wherein the inner surface of the second skirt is coated by a thromboresistant material.

Example 59. The prosthetic valve assembly of any example herein, particularly example 58, wherein the thromboresistant material comprises at least one of: TPU, a hydrogel, Heparin, Silicone, and/or combinations thereof.

Example 60. The prosthetic valve assembly of any example herein, particularly example 56, wherein the inner surface of the second skirt is configured to elute a therapeutic agent.

Example 61. The prosthetic valve assembly of any example herein, particularly example 60, wherein the therapeutic agent eluted from the second skirt comprises at least one of: Batimastat, Heparin, Sirolimus, Paclitaxel, and/or Everolimus.

Example 62. The prosthetic valve assembly of any example herein, particularly any one of examples 7 to 61, wherein an outer surface of the first skirt is thromboresistant.

Example 63. The prosthetic valve assembly of any example herein, particularly example 62, wherein the outer surface of the first skirt comprises one or more of: PTFE, ePTFE, and/or UHMWPE.

Example 64. The prosthetic valve assembly of any example herein, particularly example 62, wherein the outer surface of the first skirt is coated by a thromboresistant material.

Example 65. The prosthetic valve assembly of any example herein, particularly example 64, wherein the thromboresistant material comprises at least one of: TPU, a hydrogel, Heparin, Silicone, and/or combinations thereof.

Example 66. The prosthetic valve assembly of any example herein, particularly example 62, wherein the outer surface of the first skirt is configured to elute a therapeutic agent.

Example 67. The prosthetic valve assembly of any example herein, particularly example 66, wherein the therapeutic agent eluted from the first skirt comprises at least one of: Batimastat, Heparin, Sirolimus, Paclitaxel, and/or Everolimus.

Example 68. The prosthetic valve assembly of any example herein, particularly any one of examples 7 to 67, wherein the frame is configured to elute a therapeutic agent.

Example 69. The prosthetic valve assembly of any example herein, particularly example 68, wherein the therapeutic agent eluted from the frame comprises at least one of: Batimastat, Heparin, Sirolimus, Paclitaxel, and/or Everolimus.

Example 70. The prosthetic valve assembly of any example herein, particularly any one of examples 7 to 69, wherein the prosthetic valve further comprises a valvular structure comprising a plurality of leaflets disposed inside the frame and configured to regulate flow of blood through the prosthetic valve.

Example 71. The prosthetic valve assembly of any example herein, particularly example 70, wherein the plurality of leaflets comprises three leaflets.

Example 72. A method of assembling a prosthetic valve assembly, comprising: attaching a first skirt to a frame of a prosthetic valve; releasably coupling a second skirt to the first skirt by passing at least one retaining suture through outflow loops of the first skirt and of the second skirt, and through inflow loops of the first skirt and of the second skirt; and attaching a suture coupling end of the at least one retaining suture to the frame.

Example 73. The method of any example herein, particularly example 72, wherein the second skirt is an outer skirt of the prosthetic valve assembly.

Example 74. The method of any example herein, particularly example 73, wherein the first skirt is a base skirt of the prosthetic valve, and wherein the attaching the first skirt to the frame comprises disposing the base skirt around an outer surface of the frame.

Example 75. The method of any example herein, particularly example 74, wherein the releasably coupling the second skirt to the first skirt comprises disposing the outer skirt around an outer surface of the base skirt.

Example 76. The method of any example herein, particularly example 73 or 74, wherein the attaching the first skirt to the frame comprises suturing the base skirt to the frame.

Example 77. The method of any example herein, particularly any one of examples 74 to 76, further comprising disposing an inner skirt around an inner surface of the frame, and attaching the inner skirt to the frame.

Example 78. The method of any example herein, particularly example 77, wherein the attaching the inner skirt to the frame comprises suturing the inner skirt to the frame.

Example 79. The method of any example herein, particularly any one of examples 73 to 78, wherein the releasably coupling the second skirt to the first skirt is performed prior to the attaching the first skirt to the frame.

Example 80. The method of any example herein, particularly example 73, wherein the first skirt is an inner skirt of the prosthetic valve, and wherein the attaching the first skirt to the frame comprises disposing the inner skirt around an inner surface of the frame.

Example 81. The method of any example herein, particularly example 80, wherein the attaching the first skirt to the frame comprises suturing the inner skirt to the frame.

Example 82. The method of any example herein, particularly any one of examples 72 to 81, wherein the attaching the suture coupling end to the frame comprises forming a knot tied to the frame.

Example 83. The method of any example herein, particularly any one of examples 72 to 82, wherein the attaching the suture coupling end to the frame comprises axially offsetting the suture coupling end from the second skirt.

Example 84. The method of any example herein, particularly any one of examples 72 to 83, wherein the attaching the suture coupling end to the frame comprises attaching the suture coupling end to a portion of the frame between an outflow end of the second skirt and an outflow end of the frame.

Example 85. The method of any example herein, particularly any one of examples 72 to 84, wherein the coupling the second skirt to the first skirt comprises circumferentially positioning the second skirt and the first skirt relative to each other such that the inflow loops of the first skirt and of the second skirt are arranged in an alternating manner around the circumference of the frame, and such that the outflow loops of the first skirt and of the second skirt arranged in an alternating manner around the circumference of the frame.

Example 86. The method of any example herein, particularly any one of examples 72 to 85, wherein the at least one retaining suture comprises a single retaining suture.

Example 87. The method of any example herein, particularly example 86, wherein the releasably coupling the second skirt to the first skirt further comprises extending the retaining suture along a distally extending suture offset section from the suture coupling end toward the outflow loops of the first and the second skirts, prior to passing the retaining suture through the outflow loops.

Example 88. The method of any example herein, particularly example 87, wherein the coupling the second skirt to the first skirt further comprises, subsequent to the passing the retaining suture through the outflow loops and prior to the passing the retaining suture through the inflow loops, extending the retaining suture along a distally extending suture crossing section between the outflow loops of the first and the second skirts and the inflow loops of the first and the second skirts.

Example 89. The method of any example herein, particularly example 88, wherein the coupling the second skirt to the first skirt further comprises, subsequent to the passing the retaining suture through the inflow loops, extending the retaining suture from the inflow loops of the first and the second skirts, along a proximally extending suture crossing section and along a proximally extending suture offset section continuous therewith, toward the suture coupling end.

Example 90. The method of any example herein, particularly example 89, wherein the distally extending suture crossing section and the proximally extending suture crossing section are formed to diagonally extend at an angular orientation relative to a longitudinal axis of the prosthetic valve.

Example 91. The method of any example herein, particularly example 90, wherein the angular orientation is between 30 and 60 degrees.

Example 92. The method of any example herein, particularly example 90, wherein the angular orientation is between 40 and 50 degrees.

Example 93. The method of any example herein, particularly example 90, wherein the angular orientation is about 45 degrees.

Example 94. The method of any example herein, particularly any one of examples 90 to 93, wherein the distally extending suture offset section and the proximally extending suture offset section extend at the same angular orientation as that of the distally extending suture crossing section and the proximally extending suture crossing section.

Example 95. The method of any example herein, particularly any one of examples 72 to 85, wherein the at least one retaining suture comprises a first retaining suture and a second retaining suture.

Example 96. The method of any example herein, particularly example 95, wherein the releasably coupling the second skirt to the first skirt further comprises extending the first retaining suture along a suture offset section thereof from its suture coupling end toward the outflow loops of the first and the second skirts, prior to the passing the retaining suture through the outflow loops.

Example 97. The method of any example herein, particularly example 96, wherein the releasably coupling the second skirt to the first skirt further comprises, subsequent to the passing the retaining suture through the outflow loops, extending the first retaining suture along another suture offset section thereof from the outflow loops of the first and the second skirts towards the suture coupling end of the first retaining suture.

Example 98. The method of any example herein, particularly example 97, wherein the releasably coupling the second skirt to the first skirt further comprises extending the second retaining suture along a suture offset section thereof and a suture crossing section continuous therewith, from the suture coupling end of the second retaining suture toward the inflow loops of the first and the second skirts, prior to the passing the retaining suture through the inflow loops.

Example 99. The method of any example herein, particularly example 98, wherein the releasably coupling the second skirt to the first skirt further comprises, subsequent to the passing the retaining suture through the inflow loops, extending the second retaining suture along another suture crossing section thereof and another suture offset section continuous therewith, from the inflow loops of the first and the second skirts towards the suture coupling end of the second retaining suture.

Example 100. The method of any example herein, particularly example 99, wherein the suture crossing sections of the second retaining suture are formed to diagonally extend at an angular orientation relative to a longitudinal axis of the prosthetic valve.

Example 101. The method of any example herein, particularly example 100, wherein the angular orientation is between 30 and 60 degrees.

Example 102. The method of any example herein, particularly example 100, wherein the angular orientation is between 40 and 50 degrees.

Example 103. The method of any example herein, particularly example 100, wherein the angular orientation is about 45 degrees.

Example 104. A method of explanting a prosthetic valve, comprising: accessing an implantation site of a prosthetic valve assembly, the prosthetic valve assembly comprising the prosthetic valve having a first skirt attached to a frame, and a second skirt releasably coupled to the first skirt by at least one retaining suture which is attached to the frame at a suture coupling end and extends through one or more inflow loops of the first and the second skirts, and through one or more outflow loops of the first and the second skirts; cutting the at least one retaining suture by a cutting tool; grasping a loose end of the at least one retaining and pulling it so as to withdraw the at least one retaining suture from the one or more outflow loops of the first and the second skirts and from the one or more inflow loops of the first and the second skirts, thereby separating the prosthetic valve from the second skirt; and removing the prosthetic valve from the implantation site.

Example 105. The method of any example herein, particularly example 104, wherein the second skirt is an outer skirt of the prosthetic valve assembly.

Example 106. The method of any example herein, particularly example 105, wherein the first skirt is a base skirt disposed around an outer surface of the frame and coupled to the frame, and wherein the outer skirt is disposed around the base skirt.

Example 107. The method of any example herein, particularly any one of examples 105 to 106, wherein the prosthetic valve further comprises an inner skirt disposed around an inner surface of the frame and coupled to the frame.

Example 108. The method of any example herein, particularly example 105, wherein the first skirt is an inner skirt disposed around an inner surface of the frame and coupled to the frame, and wherein the outer skirt is disposed around an outer surface of the frame.

Example 109. The method of any example herein, particularly any one of examples 104 to 108, wherein cutting comprises cutting a suture offset section of the at least one retaining suture, extending between the suture coupling end and the second skirt.

Example 110. The method of any example herein, particularly any one of examples 104 to 109, wherein the at least one retaining suture comprises a first retaining suture extending through the inflow loops of the first and the second skirts, but not through the outflow loops of the first and the second skirts, and a second retaining suture extending through the outflow loops of the first and the second skirts, but not through the inflow loops of the first and the second skirts.

Example 111. The method of any example herein, particularly example 110, wherein the cutting comprises cutting the first retaining suture and cutting the second retaining suture.

Example 112. The method of any example herein, particularly example 111, wherein the grasping comprises grasping a loose end of the first retaining suture and pulling it so as to withdraw the first retaining suture from the one or more outflow loops of the first and the second skirts, and grasping a loose end of the second retaining suture and pulling it so as to withdraw the second retaining suture from the one or more inflow loops of the first and the second skirts.

Example 113. A prosthetic valve kit comprising: a prosthetic valve having a longitudinal axis and comprising: an annular frame movable between a radially compressed and a radially expanded configuration; and an outer skirt disposed around an outer surface of the annular frame and coupled to the annular frame; and a cutting member comprising at least one elongated segment, each extending along the annular frame from a proximal end portion of the elongated segment to a distal end portion of the elongated segment, the at least one elongated segment disposed radially outward to the annular frame; wherein the cutting member is electrically conductive.

Example 114. The prosthetic valve kit of any example herein, particularly example 113, wherein the cutting member further comprises a circumferential segment extending from the distal end portion of the elongated segment.

Example 115. The prosthetic valve kit of any example herein, particularly example 114, wherein the circumferential segment defines a plane that is generally perpendicular to an axis defined by the elongated segment.

Example 116. The prosthetic valve kit of any example herein, particularly example 114, wherein the circumferential segment defines a plane that is generally perpendicular to a longitudinal axis of the annular frame.

Example 117. The prosthetic valve kit of any example herein, particularly any one of examples 114-116, wherein the circumferential segment is shaped as a portion of an ellipse.

Example 118. The prosthetic valve kit of any example herein, particularly any one of examples 114-116, wherein the circumferential segment is shaped as a full ellipse.

Example 119. The prosthetic valve kit of any example herein, particularly any one of examples 114-118, wherein the circumferential segment is a closed shape.

Example 120. The prosthetic valve kit of any example herein, particularly any one of examples 114-118, wherein the circumferential segment is an open shape.

Example 121. The prosthetic valve kit of any example herein, particularly any one of examples 114-120, wherein the at least one elongated segment comprises a plurality of elongated segments, each of the plurality of elongated segments extending proximally from a respective portion of the circumferential segment.

Example 122. The prosthetic valve kit of any example herein, particularly example 113, wherein the at least one elongated segment comprises a plurality of elongated segments.

Example 123. The prosthetic valve kit of any example herein, particularly example 121 or 122, wherein the proximal end portion of each of the plurality of elongated segments is bent towards the longitudinal axis of the prosthetic valve.

Example 124. The prosthetic valve kit of any example herein, particularly any one of examples 113-120, wherein the proximal end portion of the at least one elongated segment is bent towards the longitudinal axis of the prosthetic valve.

Example 125. The prosthetic valve kit of any example herein, particularly any one of examples 113-120, wherein the distal end portion of the at least one elongated segment is bent towards the longitudinal axis of the prosthetic valve.

Example 126. The prosthetic valve kit of any example herein, particularly any one of examples 113-125, wherein the cutting member further comprises a first terminal extending from the proximal end portion of the at least one elongated segment.

Example 127. The prosthetic valve kit of any example herein, particularly example 126, wherein the cutting member further comprises a second terminal extending from the distal end portion of the at least one elongated segment.

Example 128. The prosthetic valve kit of any example herein, particularly example 126 or 127, wherein the first terminal is generally rectangular shaped.

Example 129. The prosthetic valve kit of any example herein, particularly any one of examples 126-128, wherein the second terminal is generally rectangular shaped.

Example 130. The prosthetic valve kit of any example herein, particularly any one of examples 127-129, further comprising a power transfer device, the power transfer device providing an electric current path to the cutting member, wherein the power transfer device is configured to be attached to the first terminal or the second terminal.

Example 131. The prosthetic valve kit of any example herein, particularly example 130, wherein the power transfer device is configured to alternately be attached to the first terminal and the second terminal.

Example 132. The prosthetic valve kit of any example herein, particularly example 130 or 131, wherein the power transfer device comprises an alligator clip.

Example 133. The prosthetic valve kit of any example herein, particularly any one of examples 113-129, further comprising a power transfer device, the power transfer device providing an electric current path to the cutting member.

Example 134. The prosthetic valve kit of any example herein, particularly example 132, wherein the power transfer device comprises an alligator clip.

Example 135. The prosthetic valve kit of any example herein, particularly any one of examples 113-134, wherein the elongated segment is disposed between the annular frame and the outer skirt.

Example 136. The prosthetic valve kit of any example herein, particularly example 135, wherein the prosthetic valve further comprises at least one suture, the at least one suture securing the outer skirt to the annular frame.

Example 137. The prosthetic valve kit of any example herein, particularly example 136, wherein the at least one suture surrounds a portion of the annular frame.

Example 138. The prosthetic valve kit of any example herein, particularly example 137, further comprising an inner skirt surrounding an inner surface of the annular frame, wherein the at least one suture is secured to the inner skirt.

Example 139. The prosthetic valve kit of any example herein, particularly any one of examples 113-138, wherein the elongated segment is disposed radially outward to an outer surface of the outer skirt.

Example 140. The prosthetic valve kit of any example herein, particularly any one of examples 113-139, wherein at least a portion of the elongated segment is covered with an electrically insulated surface.

Example 141. A prosthetic valve assembly comprising: the prosthetic valve kit of any example herein, particularly any one of examples 113-140; and an electric current source configured to provide an electric current to the cutting member.

Example 142. The prosthetic valve assembly of any example herein, particularly example 141, wherein the provided electric current is an alternating current.

Example 143. The prosthetic valve assembly of any example herein, particularly example 142, wherein a frequency of the alternating current is a radio-frequency.

Example 144. A method of cutting an outer skirt of a prosthetic valve having a longitudinal axis and comprising an annular frame, the outer skirt disposed around an outer surface of the annular frame and coupled to the annular frame, wherein the method comprises: providing an electric current to a cutting member, the cutting member comprising at least one elongated segment extending along the annular frame from a proximal end portion of the elongated segment to a distal end portion of the elongated segment, the at least one elongated segment disposed radially outward to the annular frame; and axially pulling the at least one cutting member.

Example 145. The method of any example herein, particularly example 144, wherein the cutting member further comprises a circumferential segment extending from the distal end portion of the elongated segment.

Example 146. The method of any example herein, particularly example 145, wherein the circumferential segment defines a plane that is generally perpendicular to an axis defined by the elongated segment.

Example 147. The method of any example herein, particularly example 145, wherein the circumferential segment defines a plane that is generally perpendicular to the longitudinal axis of the prosthetic valve.

Example 148. The method of any example herein, particularly any one of examples 145-147, wherein the circumferential segment is shaped as a portion of an ellipse.

Example 149. The method of any example herein, particularly any one of examples 145-147, wherein the circumferential segment is shaped as a full ellipse.

Example 150. The method of any example herein, particularly any one of examples 145-148, wherein the circumferential segment is a closed shape.

Example 151. The method of any example herein, particularly any one of examples 145-148, wherein the circumferential segment is an open shape.

Example 152. The method of any example herein, particularly any one of examples 145-151, wherein the at least one elongated segment comprises a plurality of elongated segments, each of the plurality of elongated segments extending proximally from a respective portion of the circumferential segment.

Example 153. The method of any example herein, particularly example 144, wherein the at least one elongated segment comprises a plurality of elongated segments.

Example 154. The method of any example herein, particularly example 152 or 153, wherein the proximal end portion of each of the plurality of elongated segments is bent towards the longitudinal axis of the prosthetic valve.

Example 155. The method of any example herein, particularly any one of examples 144-151, wherein the proximal end portion of the at least one elongated segment is bent towards the longitudinal axis of the prosthetic valve.

Example 156. The method of any example herein, particularly any one of examples 144-151, wherein the distal end portion of the at least one elongated segment is bent towards the longitudinal axis of the prosthetic valve.

Example 157. The method of any example herein, particularly any one of examples 144-156 wherein the cutting member further comprises a first terminal extending from the proximal end portion of the at least one elongated segment.

Example 158. The method of any example herein, particularly example 144, wherein the cutting member further comprises a second terminal extending from the distal end portion of the at least one elongated segment.

Example 159. The method of any example herein, particularly example 157 or 158, wherein the first terminal is generally rectangular shaped.

Example 160. The method of any example herein, particularly any one of examples 157-159, wherein the second terminal is generally rectangular shaped.

Example 161. The method of any example herein, particularly any one of examples 158-160, further comprising attaching a power transfer device to the first terminal or the second terminal, the electric current provided to the cutting member via the power transfer device.

Example 162. The method of any example herein, particularly example 161, wherein the power transfer device is alternately attached to the first terminal and the second terminal.

Example 163. The method of any example herein, particularly example 161 or 162, wherein the power transfer device comprises an alligator clip.

Example 164. The method of any example herein, particularly any one of examples 144-160, further comprising attaching a power transfer device to the cutting member, the electric current provided to the cutting member via the power transfer device.

Example 165. The method of any example herein, particularly example 164, wherein the power transfer device comprises an alligator clip.

Example 166. The method of any example herein, particularly any one of examples 144-151, wherein the elongated segment is disposed between the annular frame and the outer skirt, and wherein heat generated by the provided electric current cuts the outer skirt from the annular frame.

Example 167. The method of any example herein, particularly example 166, wherein the prosthetic valve further comprises at least one suture, the at least one suture securing the outer skirt to the annular frame, the generated heat configured to cut the at least one suture.

Example 168. The method of any example herein, particularly example 167, wherein the at least one suture surrounds a portion of the annular frame.

Example 169. The method of any example herein, particularly example 167, further comprising an inner skirt surrounding an inner surface of the annular frame, wherein the at least one suture is secured to the inner skirt.

Example 170. The method of any example herein, particularly any one of examples 144-165, wherein the elongated segment is disposed radially outward to an outer surface of the outer skirt, and wherein heat generated by the provided electric current cuts the outer skirt from surrounding tissue.

Example 171. The method of any example herein, particularly any one of examples 144-170, wherein at least a portion of the elongated segment is covered with an electrically insulated surface.

Example 172. The method of any example herein, particularly any one of examples 144-171, wherein the provided electric current is an alternating current.

Example 173. The method of any example herein, particularly example 172, wherein a frequency of the alternating current is a radio-frequency.

Example 174. The method of any example herein, particularly any one of examples 144-173, wherein the providing the electric current to a cutting member comprises providing the electric current from an electric current source which is configured, responsive to a user input, to alternately provide the electric current and not provide the respective electric current.

Example 175. A prosthetic valve comprising: an annular frame movable between a radially compressed and a radially expanded configuration; a first skirt coupled to the frame; and at least one skirt removal device.

Example 176. The prosthetic valve of any example herein, particularly example 175, wherein the at least one skirt removal device comprises a suture releasably coupled to the annual frame and the first skirt, and wherein decoupling the at least one skirt removal device from the annual frame is configured to allow separation between the annual frame and the first skirt.

Example 177. The prosthetic valve of any example herein, particularly example 175, wherein the at least one skirt removal device comprises a cutting member configured to separate the first skirt from the annular frame.

Example 178. The prosthetic valve of any example herein, particularly example 177, wherein the cutting member utilizes RF energy to separate the first skirt from the annular frame.

Example 179. The prosthetic valve of any example herein, particularly any one of examples 175-178 comprising: a second skirt coupled to the frame, wherein the second skirt is positioned radially outside of the first skirt.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve assembly comprising:
a prosthetic valve comprising:
an annular frame movable between a radially compressed and a radially expanded configuration; and
a removable skirt disposed radially outward to the frame, the removable skirt comprising one or more inflow loops extending from an inflow end of the removable skirt, and one or more outflow loops extending from an outflow end of the removable skirt; and
at least one retaining suture coupled to the frame at a suture coupling end, and extending through the one or more inflow loops of the removable skirt, and through the one or more outflow loops of the removable skirt;
wherein the one or more inflow loops of the removable skirt and the one or more outflow loops of the removable skirt are arranged around the circumference of the frame; and
wherein the removable skirt is configured to be released from the frame upon withdrawal of the at least one retaining suture from the one or more inflow loops of the removable skirt and from the one or more outflow loops of the removable skirt.

2. The prosthetic valve assembly of claim 1, wherein the removable skirt is an outer skirt.

3. The prosthetic valve assembly of claim 1, wherein the one or more inflow loops of the removable skirt comprises a plurality of inflow loops of the removable skirt, arranged around the circumference of the frame, and wherein the one or more outflow loops of the removable skirt comprises a plurality of outflow loops of the removable skirt, arranged around the circumference of the frame.

4. The prosthetic valve assembly of claim 3, wherein the at least one retaining suture is weaved around struts of the frame between adjacent inflow loops of the plurality of inflow loops of the removable skirt, and around struts of the frame between adjacent outflow loops of the plurality of outflow loops of the removable skirt.

5. The prosthetic valve assembly of claim 1, wherein the prosthetic valve further comprises a first skirt disposed around the frame and coupled to the frame, the first skirt comprising one or more inflow loops extending from an inflow end of the first skirt, and one or more outflow loops extending from an outflow end of the first skirt; wherein the removable skirt is a second skirt; wherein the at least one retaining suture extends through the one or more inflow loops of the first and the second skirts, and through the one or more outflow loops of the first and the second skirts; wherein the one or more inflow loops of the first skirt and of the second skirt are arranged in an alternating manner around the circumference of the frame; wherein the one or more outflow loops of the first skirt and of the second skirt are arranged in an alternating manner around the circumference of the frame; and wherein the second skirt is configured to be released from the first skirt upon withdrawal of the at least one retaining suture from the one or more inflow loops of the first and the second skirts and from the one or more outflow loops of the first and the second skirts.

6. The prosthetic valve assembly of claim 5, wherein the second skirt is an outer skirt of the prosthetic valve assembly, wherein the inflow end of the second skirt is an outer skirt inflow end, wherein the outflow end of the second skirt is an outer skirt outflow end, wherein the one or more inflow loops of the second skirt is one or more outer skirt inflow loops, and wherein the one or more outflow loops of the second skirt is one or more outer skirt outflow loops.

7. The prosthetic valve assembly of claim 6, wherein the first skirt is a base skirt disposed around an outer surface of the frame, wherein the inflow end of the first skirt is a base skirt inflow end, wherein the outflow end of the first skirt is a base skirt outflow end, wherein the one or more inflow loops of the first skirt is one or more base skirt inflow loops, and wherein the one or more outflow loops of the first skirt is one or more base skirt outflow loops.

8. The prosthetic valve assembly of claim 6, wherein the first skirt is an inner skirt disposed around an inner surface of the frame, wherein the inflow end of the first skirt is an inner skirt inflow end, wherein the outflow end of the first skirt is an inner skirt outflow end, wherein the one or more inflow loops of the first skirt is one or more inner skirt inflow loops, and wherein the one or more outflow loops of the first skirt is one or more inner skirt outflow loops.

9. The prosthetic valve assembly of claim 5, wherein the suture coupling end comprises a knot at which the at least one retaining suture is tied to the frame.

10. A method of assembling a prosthetic valve assembly, comprising:
attaching a first skirt to a frame of a prosthetic valve;
releasably coupling a second skirt to the first skirt by passing at least one retaining suture through outflow loops of the first skirt and of the second skirt, and through inflow loops of the first skirt and of the second skirt; and
attaching a suture coupling end of the at least one retaining suture to the frame.

11. The method of claim 10, wherein the second skirt is an outer skirt of the prosthetic valve assembly.

12. The method of claim 11, wherein the first skirt is a base skirt of the prosthetic valve, and wherein the attaching the first skirt to the frame comprises disposing the base skirt around an outer surface of the frame.

13. The method of claim 12, further comprising disposing an inner skirt around an inner surface of the frame, and attaching the inner skirt to the frame.

14. The method of claim 11, wherein the first skirt is an inner skirt of the prosthetic valve, and wherein the attaching the first skirt to the frame comprises disposing the inner skirt around an inner surface of the frame.

15. The method of claim 10, wherein the coupling the second skirt to the first skirt comprises circumferentially positioning the second skirt and the first skirt relative to each other such that the inflow loops of the first skirt and of the second skirt are arranged in an alternating manner around the circumference of the frame, and such that the outflow loops of the first skirt and of the second skirt arranged in an alternating manner around the circumference of the frame.

16. A method of explanting a prosthetic valve, comprising:
accessing an implantation site of a prosthetic valve assembly, the prosthetic valve assembly comprising the prosthetic valve having a first skirt attached to a frame, and a second skirt releasably coupled to the first skirt by at least one retaining suture which is attached to the frame at a suture coupling end and extends through one or more inflow loops of the first and the second skirts, and through one or more outflow loops of the first and the second skirts;
cutting the at least one retaining suture by a cutting tool;
grasping a loose end of the at least one retaining and pulling it so as to withdraw the at least one retaining suture from the one or more outflow loops of the first and the second skirts and from the one or more inflow loops of the first and the second skirts, thereby separating the prosthetic valve from the second skirt; and
removing the prosthetic valve from the implantation site.

17. The method of claim 16, wherein the second skirt is an outer skirt of the prosthetic valve assembly.

18. The method of claim 17, wherein the first skirt is a base skirt disposed around an outer surface of the frame and coupled to the frame, and wherein the outer skirt is disposed around the base skirt.

19. The method of claim 17, wherein the first skirt is an inner skirt disposed around an inner surface of the frame and coupled to the frame, and wherein the outer skirt is disposed around an outer surface of the frame.

20. The method of claim 16, wherein cutting comprises cutting a suture offset section of the at least one retaining suture, extending between the suture coupling end and the second skirt.
